(19) United States
(12) Patent Application Publication
(10) Pub. No.: US 2020/0289441 A1
(43) Pub. Date: Sep. 17, 2020

(54) METHODS FOR PROMOTING HAIR GROWTH USING VALPROIC ACID

(71) Applicant: FOLLICA, INC., Boston, MA (US)

(72) Inventors: Jason Venkat Bhardwaj, Boston, MA (US); Scott C. Kellogg, Mattapoisett, MA (US); Craig Russo, Newton, MA (US)

(21) Appl. No.: 16/646,445

(22) PCT Filed: Sep. 13, 2018
(86) PCT No.: PCT/US2018/050895

§ 371 Date: Mar. 11, 2020

Related U.S. Application Data
(60) Provisional application No. 62/558,778, filed on Sep. 14, 2017.

(51) Int. Cl. A61K 31/19; A61K 9/00; A61K 9/70; A61K 45/06; A61P 17/14

(52) U.S. Cl. CPC A61K 31/19; A61K 9/0021; A61P 17/14; A61K 9/0053; A61K 45/06; A61K 9/7023

(57) ABSTRACT

The invention relates to methods of treating baldness, treating alopecia, promoting hair growth, and/or promoting hair follicle development and/or activation or stimulation on an area of the skin of a subject (for example, a human) by subjecting said area of the skin to integumental perturbation; and administration of a pharmaceutical composition (e.g., a pharmaceutical composition comprising valproic acid or a pharmaceutically acceptable salt thereof).

### 1. CROSS-REFERENCE TO RELATED APPLICATION

This application claims the benefit of priority to U.S. Provisional Patent Application No. 62/558,778, filed on Sep. 14, 2017, which is incorporated herein by reference in its entirety for all purposes.

### 2. INTRODUCTION

The invention relates to methods of treating baldness, treating alopecia, promoting hair growth, and/or promoting hair follicle development and/or activation on an area of the skin of a subject (for example, a human subject) by subjecting said area of the skin to integumental perturbation and administering to said area valproic acid or a pharmaceutically acceptable salt thereof (e.g., a pharmaceutical composition comprising valproic acid or a pharmaceutically acceptable salt, isotopic variant, or solvate thereof).

### 3. BACKGROUND

Follicular neogenesis is defined as the generation of new hair follicles (HF) after birth. Humans are born with a full complement of HF, which can change in size and growth characteristics as in early baldness or can ultimately degenerate and disappear as in late stages of baldness or in permanent scarring (cicatricial) alopecias. Because of limited effective treatment options, there is substantial interest among individuals for novel, safe and effective treatments for hair loss, including those that lead to hair follicle neogenesis, resulting in visible hair.

Hair loss treatment can lead to hair follicle neogenesis, resulting in visible hair. Such methods are described for example in International Patent Application Publication No. WO 2012/078649 the contents of which is incorporated by reference in its entirety.

Methods and devices for integumental perturbation are described, for example in International Patent Application Publication No. WO 2017/054009, which is incorporated by reference in its entirety. A drug applicator device may be used for multiple drug application purposes such as applying a hair growth compound to the skin of a subject. A drug applicator device that can be used is also described in International Patent Application Publication No. WO 2017/054009.

Valproic acid is a known wnt agonist and inhibitor of GSK3Beta, which stabilizes beta catenin. See Wiltse et al., “Mode of action: inhibition of histone deacetylase, altering wnt-dependent gene expression, and regulation of beta-catenin-developmental effects of valproic acid.” Critical Reviews in Toxicology, (2005), 35, 8-9, 727-738. Valproic acid may also affect GABA levels, block voltage-gated sodium channels, and inhibit histone deacetylases. Topical valproic acid has been shown to increase total hair counts. See Jo et al., “Topical valproic acid increases the hair count in male patients with androgenetic alopecia: A randomized, comparative, clinical feasibility study using phototrichogram analysis.” Journal of Dermatology, (2014); 41: 285-291.

### 4. SUMMARY OF THE INVENTION

In an aspect, provided herein is a method for promoting hair growth in a human subject, wherein the method comprises: integumental perturbation of an area of the skin of the human subject where hair growth is desired; and administering a pharmaceutical composition comprising valproic acid or a pharmaceutically acceptable salt, isotopic variant, or solvate thereof. In some embodiments, the area of the skin of the human subject where hair growth is desired is the scalp of the human subject or a part of the scalp of the human subject.

In some embodiments, the valproic acid or a pharmaceutically acceptable salt thereof is present in an amount of 0.5-30% wt % based on the total weight of the composition. In some embodiments, the valproic acid or a pharmaceutically acceptable salt thereof is present in an amount of 2.0-25% wt % based on the total weight of the composition. The terms “composition”, “valproic acid composition”, “pharmaceutical composition”, “pharmaceutical composition comprising valproic acid or a pharmaceutically acceptable salt thereof are used interchangeably to refer to the valproic acid composition.

In some embodiments, the valproic acid composition is administered once reepithelialization is completed, or 1, 2, 3, 4, 5, 6, 7, 8, 9, 10, 11, 12, 13, 14, 15, or 16 weeks after integumental perturbation. In some embodiments, the valproic acid composition is administered once reepithelialization is completed, or at least 2, 4, 8, 10, 12, 14, 16, 18, 20, 22, or 24 hours after integumental perturbation. In some embodiments, the valproic acid composition is administered once reepithelialization is completed, or at least 1, 2, 3, 4, 5, 6, 7 days after integumental perturbation. In some embodiments, the valproic acid composition is first administered 1 day after integumental perturbation. In some embodiments, the valproic acid composition is administered before and after integumental perturbation. In some embodiments, the integumental perturbation is performed after 24 or more hours of valproic acid composition administration.

In some embodiments, the valproic acid composition is administered for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. In some embodiments, the valproic acid composition is administered 1, 2, 3, or 4 times every 1, 2, 3, or 4 days for a period of 1, 2, 3, 4, 5, 6, or 7 days. In some embodiments, the valproic acid composition is administered 1 or 2 times every 1 day for a period of 1, 2, 3, 4, 5, 6, or 7 days. In some embodiments, the valproic acid composition is administered every 12 hours for a period of 1, 2, 3, 4, 5, 6, or 7 days. In some embodiments, the valproic acid composition administration schedule is repeated one or more times, for example, repeated 1, 2, 3, 4, 5, 6, 7, 8, 9, 10, 11, 12, 13, 14, 15, 16, 17, 18, 19, 20, 21, 22, 23, 24, 25, 26, 27, 28, or 29 times. In some embodiments, the integumental perturbation is performed once a week for 1, 2, 3, 4, 5, 6, 7, 8, 9, 10, 11, 12, 13, 14, 15, or 16 weeks. In some embodiments, the integumental perturbation is performed every other week, e.g., twice a month, e.g., bi-weekly, for 1, 2, 3, or 4 months. In some embodiments, the integumental perturbation is performed once a month for 1, 2, 3, or 4 months. In any of these embodiments, the integumental perturbation is performed for longer than 4 months. In some embodiments, any of the integumental perturbation and valproic acid composition administration schedules/cycles is repeated one or more times, for example, repeated 1, 2, 3, 4, 5, 6, 7, 8, 9, 10, 11, 12, 13, 14, 15, 16, 17, 18, 19, 20, 21, 22, 23, 24, 25, 26, 27, 28, or 29 times.

In some embodiments, the integumental perturbation penetrates the skin to a depth of 500 μm to 2.5 mm.

In some embodiments, the integumental perturbation is performed by dermabrasion, laser, or controlled integumental perturbation. In some embodiments, the integumental perturbation is performed by dermabrasion. In some embodiments, the integumental perturbation is performed until pinpoint bleeding occurs.

In some embodiments, the integumental perturbation is performed by a needling device or drug applicator device. In some embodiments, the integumental perturbation is performed by micro-needling.

In some embodiments, the maximum depth of the integumental perturbation is 500, 600, 700, 800, 900, 1000, 1100, 1200, 1300, 1400, 1500, 1600, 1700, 1800, 1900, 2000, 2100, 2200, 2300, 2400 or 2500 μm. In some embodiments, the maximum depth of the integumental perturbation is 700, 800, 900, or 1000 μm. In some embodiments, the maximum depth of the integumental perturbation is 800 μm.

In some embodiments, the composition is administered transdermally.

In some embodiments, the composition is administered subcutaneously or externally applied to the skin of the subject, e.g., topically.

In some embodiments, the composition is administered orally.

In some embodiments, the composition is a cream, gel, lotion, emulsion, suspension, oil, non-aqueous solution, aqueous solution, or drop. In some embodiments, the composition is a gel, hydrogel, emulsion, solution, suspension, cream, ointment, dusting powder, dressing, elixir, lotion, suspension, tincture, paste, powder, crystal, foams film, aerosol, irrigation, spray, suppository, stick, bar, ointment, bandage, wound dressing, microdermabrasion or dermabrasion particle, drop, transdermal patch, or dermal patch. In some embodiments, the composition is an aqueous formulation, non-aqueous formulation, ointment, or cream.

In some embodiments, the composition is administered for 1, 2, 3 or more months.

In some embodiments, the composition is administered by a drug applicator device or cartridge. In some embodiments, the cartridge contains multiple compounds for simultaneous delivery.

In some embodiments, the composition is administered as part of an article of manufacture. In some embodiments, the article of manufacture is a wound healing dressing. In some embodiments, the wound healing dressing is a bandage.

In some embodiments, the method comprises administering a hair growth promoting agent. In some embodiments, the method comprises administering an additional active ingredient.

In some embodiments, the method comprises administering an additional active ingredient before, after, or concurrently with administration of the pharmaceutical composition comprising valproic acid or a pharmaceutically acceptable salt, isotopic variant, or solvate thereof.

In some embodiments, at 3 months after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more vellus hair compared to immediately before the integumental perturbation. In some embodiments, at 3 months after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more non-vellus hair compared to immediately before the integumental perturbation.

In some embodiments, at 3 months after the integumental perturbation, the area of the skin, e.g., scalp or face, of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more hair growth or hair thickness compared to immediately before the integumental perturbation.

In some embodiments, the pharmaceutical composition is administered 1, 2, 3, or more weeks after integumental perturbation. In some embodiments, the pharmaceutical composition is administered 1 week after integumental perturbation. In some embodiments, the pharmaceutical composition is administered 2 or more weeks after integumental perturbation.

In some embodiments, the needling device comprises: a sheath assembly comprising a needle array; and a main unit comprising a motor for driving the needle array, wherein the main unit is configured to be fully encapsulated within the sheath assembly so that all parts of the main unit are protected from the outside environment. In some embodiments, the needling device comprises a needling adaptor arranged on a rectangular needle holder in two parallel or substantially parallel rows. In some embodiments, the needling device is a micropen. In an aspect, provided herein is a course of therapy for promoting hair growth in a human subject, wherein the course comprises performing the method described herein one or more times. In some embodiments, the course occurs over 1, 2, or 3 months. In some embodiments, the course occurs over 4 or more months. In some embodiments, the course comprises performing integumental perturbation and administering the pharmaceutical composition comprising valproic acid or a pharmaceutically acceptable salt thereof 3, 6, or 12 times. In some embodiments, the course comprises performing integumental perturbation monthly, biweekly, or weekly. In some embodiments, the course comprises administering the pharmaceutical composition comprising valproic acid or a pharmaceutically acceptable salt thereof 1, 2, 3, or 4 times every day for 1, 2, 3, 4, 5, 6, or 7 days. In some embodiments, the course comprises administering the pharmaceutical composition comprising valproic acid or a pharmaceutically acceptable salt thereof 1 or 2 times every day for 1, 2, 3, or 4 weeks. In some embodiments, the course comprises administering the pharmaceutical composition comprising valproic acid or a pharmaceutically acceptable salt thereof 1 or 2 times every day for 1, 2, 3, 4 or more days. In some embodiments, the course comprises administering the pharmaceutical composition comprising valproic acid or a pharmaceutically acceptable salt thereof 1 or 2 times every day for 1, 2, 3, 4, 5, 6, 7, 8, 9, 10, 11, 12, 13, 14, 15, or 16 weeks. In some embodiments, the pharmaceutical composition comprising valproic acid or a pharmaceutically acceptable salt thereof is administered after at least 24 hours following integumental perturbation. In some embodiments, the integumental perturbation is performed after at least 24 hours following administration of a pharmaceutical composition comprising valproic acid or a pharmaceutically acceptable salt thereof.

### 5. BRIEF DESCRIPTION OF THE FIGURES

FIG. 1A and 1B depict diagrams showing one embodiment of the disclosure in which an area to be treated is contacted using a needling device and/or adaptor described herein according to the “mow the lawn” technique. Two perpendicular passes are performed (e.g., horizontal and vertical). In one specific embodiment, the translation speed is 2 cm/s.

FIG. 2 depicts a diagram of one embodiment of the disclosure in which an area to be treated is contacted using a needling device and/or adaptor described herein using two non-overlapping passes (horizonal and vertical). The diagram assumes even spacing of follicles, 2.2 follicles per follicle cluster for 100 follicle clusters per 100 mm\hat{ }2. The follicle diameter is 250 um. In one embodiment, the drag distance is 76 um and the depth is 0.8 mm.

FIG. 3 depicts a diagram relating to one aspect of a method of the disclosure in which an area to be treated is contacted using a needling device and/or adaptor described herein, showing three diagrams of potential needle strikes. In the middle representation the device is perpendicular to the skin, resulting in the desired even penetration depth. In the representation to the right and the left, the device is not perpendicular to the skin, which can result in uneven penetration depth.

FIG. 4 depicts a schematic relating to one embodiment of the disclosure in which the area to be treated is contacted using a needling device and/or adaptor described herein, showing the initiation of a pass. In the upper panel, the result of a “plane landing” approach, i.e., a gliding start for the approach is illustrated. In the lower panel movement is not immediately commenced upon contact, resulting in a very high density at the site of stroke initiation.

FIG. 5 depicts a schematic relating to one embodiment of the disclosure in which the area to be treated is contacted using a needling device and/or adaptor described herein, showing how depth may be impacted by “out of tolerance” pressure. Consistent, light pressure allows the device to glide along the skin surface.

### 6. DETAILED DESCRIPTION OF THE INVENTION

Before explaining at least one embodiment of the invention in detail, it is to be understood that the invention is not limited in its application to the details of construction and to the arrangements of the components set forth in the following description or illustrated in the drawings.

Described herein are methods for promoting hair growth in a human subject, wherein the method comprises: integumental perturbation (e.g., integumental perturbation as described in Section 6.2) of an area of interest, e.g., the bald scalp of the human subject (e.g., a human subject as described in Section 6.4.2); and administering valproic acid or a pharmaceutically acceptable salt, isotopic variant, or solvate thereof (e.g., valproic acid as described in Section 6.1.2). In some embodiments, the integumental perturbation is performed with a micro-needling device as described in more detail in Section 6.2.1.

Integumental perturbation and administration of valproic acid can be performed at the same time, immediately preceding each other, or with a delay of 1, 2, 3, 4, 5, or 6 days; 1, 2, or 3 weeks, 1, 2, or 3 months. For example, valproic acid or a pharmaceutically acceptable salt thereof can be administered within 1, 2, 3, 4, 5, or 6 days; 1, 2, or 3 weeks, 1, 2, or 3 months of integumental perturbation. In an embodiment, administration of valproic acid or a pharmaceutically acceptable salt thereof occurs within 1 to 3 days, 1 to 5 days, 1 to 2 weeks, 1 to 3 weeks of integumental perturbation. In certain embodiments, treatment with valproic acid or a pharmaceutically acceptable salt thereof is commenced on the same day as the integumental perturbation and is continued once, twice, three times, four times, or five times daily for 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days. In some embodiments, the administration of valproic acid or a pharmaceutically acceptable salt thereof occurs after integumental perturbation. In some embodiments, the administration of valproic acid or a pharmaceutically acceptable salt thereof occurs at least 5, 10, 15, 30, 45, 60 minutes; 1, 2, 3, 4, 6, 8, 10, 12, 14, 16, 18, 20, 24 hours; 1, 2, 3, 4, 5, 6, or 7 days after integumental perturbation. In any of these embodiments, the integumental perturbation and administration of valproic acid cycle/schedule is repeated at least once. In some embodiments, the integumental perturbation and administration of valproic acid cycle/schedule is repeated one time, two times, three times, four times, five times, six times, seven times, eight times, nine time, ten times, eleven times, twelve times, thirteen times, fourteen times, fifteen times, or sixteen times.

Also provided herein are methods of enhancing, stimulating, or increasing hair growth or enhancing or increasing the thickness of hair (in some embodiments collectively referred to herein as “promoting” hair growth or hair thickness) on an area of skin of a subject (e.g., a human), the methods comprising subjecting an affected area of the skin to integumental perturbation in combination with administration of a valproic acid or a pharmaceutically acceptable salt thereof (e.g., a pharmaceutical composition comprising valproic acid or a pharmaceutically acceptable salt thereof). In certain embodiments, the method of treating baldness or alopecia or promoting hair growth or thickness of hair results in formation of new hair follicles (“hair follicle neogenesis”), the formation of neogenic-like hair follicles, activation of existing hair follicles, reorganization of existing hair follicles, an increase in the numbers of vellus hairs, an increase in the numbers of non-vellus hairs (e.g., intermediate or terminal), and/or an increase in the numbers of terminal hairs in the treated area.

In one embodiment, the methods described herein promotes growth of hair on an area of skin of a subject. In some embodiments, the methods described herein increases the amount or thickness of hair on a treated area of skin of a subject. In some embodiments, the methods described herein results in an increase in the amount of vellus hair on a treated area of skin of a subject. In some embodiments, the methods described herein results in an increase in the amount of non-vellus hair on a treated area of skin of a subject. In some embodiments, the methods described herein results in an increase in the amount of terminal hair on a treated area of skin of a subject. In some embodiments, the methods described herein results in formation of new hair follicles (“hair follicle neogenesis”) in a treated area of skin of a subject. In certain embodiments, the methods described herein results in an increased number of hair follicles in a treated area of skin of a subject. In particular embodiments, the methods described herein results in formation of new hair follicles with vellus-sized hair shafts (i.e., hair shafts with diameters less than 30 microns in diameter) in a treated area of skin of a subject. In some embodiments, the methods described herein results in an increased number of stimulated and activated hair follicles, such as pre-existing hair follicles, in a treated area of skin of a subject. In particular embodiments, the methods described herein results in an increased number of pre-existing hair follicles with vellus-sized hair shafts in a treated area of skin of a subject. In particular embodiments, the methods described herein results in the presence and/or increased numbers of (Neogenic-Like) follicular structure (NL), Pre-Existing-Like) follicular structure (PEL), and Pre-Existing-Like, Attached) follicular structure (PELA) follicular structures. In other particular embodiments, methods described herein results in the presence and/or increased induction of hair follicle neogenesis, and/or initiated and/or increase stimulation , activation, and/or reorganization of follicular structures. In some embodiments, the methods described herein result in newly formed, newly branched and/or newly reorganized hair follicles.

In some embodiments, valproic acid or a pharmaceutically acceptable salt thereof is administered before and after integumental perturbation. In some embodiments, the pharmaceutical composition is administered once reepithelialization is completed, or 1, 2, 3, 4, 5, 6, 7, 8, 9, 10, 11, 12, 13, 14, 15, or 16 weeks after integumental perturbation.

In some embodiments, valproic acid or a pharmaceutically acceptable salt thereof is administered fora period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. In some embodiments, valproic acid or a pharmaceutically acceptable salt thereof is administered regularly (for example, once daily, twice daily, 1, 2, 3, 4, 5, 6, or 7 times weekly).

In some embodiments, the valproic acid composition is administered 1, 2, 3, or 4 times every 1, 2, 3, or 4 days for a period of 1, 2, 3, 4, 5, 6, or 7 days. In some embodiments, the valproic acid composition is administered 1 or 2 times every 1 day for a period of 1, 2, 3, 4, 5, 6, or 7 days. In some embodiments, the valproic acid composition is administered every 12 hours for a period of 1, 2, 3, 4, 5, 6, or 7 days. In some embodiments, the valproic acid composition administration schedule is repeated one or more times, for example, repeated 1, 2, 3, 4, 5, 6, 7, 8, 9, 10, 11, 12, 13, 14, 15, 16, 17, 18, 19, 20, 21, 22, 23, 24, 25, 26, 27, 28, or 29 times. In some embodiments, the integumental perturbation is performed once a week for 1, 2, 3, 4, 5, 6, 7, 8, 9, 10, 11, 12, 13, 14, 15, or 16 weeks. In some embodiments, the integumental perturbation is performed every other week, e.g., twice a month, for 1, 2, 3, or 4 months. In some embodiments, the integumental perturbation is performed once a month for 1, 2, 3, or 4 months. In any of these embodiments, the integumental perturbation is performed for longer than 4 months. In some embodiments, any of the integumental perturbation and valproic acid composition administration schedules/cycles is repeated one or more times, for example, repeated 1, 2, 3, 4, 5, 6, 7, 8, 9, 10, 11, 12, 13, 14, 15, 16, 17, 18, 19, 20, 21, 22, 23, 24, 25, 26, 27, 28, or 29 times.

The methods described herein, in some embodiments, comprises administering a pharmaceutical composition comprising valproic acid or a pharmaceutically acceptable salt thereof (e.g., a pharmaceutical composition as described in Section 6.1.3) transdermally. In some embodiments, the pharmaceutical composition is administered subcutaneously or externally applied to the skin of the subject. In some embodiments, the pharmaceutical composition is administered orally.

In some embodiments, the pharmaceutical composition is a cream, gel, lotion, emulsion, suspension, oil, non-aqueous solution, aqueous solution, or drop. In some embodiments, the pharmaceutical composition is a gel, hydrogel, emulsion, solution, suspension, cream, ointment, dusting powder, dressing, elixir, lotion, suspension, tincture, paste, powder, crystal, foams film, aerosol, irrigation, spray, suppository, stick, bar, ointment, bandage, wound dressing, microdermabrasion or dermabrasion particle, drop, transdermal patch, or dermal patch. In some embodiments, the pharmaceutical composition is an aqueous formulation, non-aqueous formulation, ointment, or cream. In some embodiments, the pharmaceutical composition is administered as part of an article of manufacture. In some embodiments, the article of manufacture is a wound healing dressing. In some embodiments, the wound healing dressing is a bandage.

The methods described herein comprises, in some embodiments, integumental perturbation and topical administration of a pharmaceutical composition that is intended to promote the growth of hair (e.g., a pharmaceutical composition comprising valproic acid). The methods described may comprise administration of an additional agent (e.g., active ingredient, e.g., hair growth-promoting agent). Exemplary additional agents are described in detail in Section 6.5. The additional agent may be administered before or after administration of a pharmaceutical composition (e.g., a pharmaceutical composition comprising valproic acid). The additional agent may be administered concurrent with (e.g., at the same time as) administration of a pharmaceutical composition (e.g., a pharmaceutical composition comprising valproic acid).

The methods described herein optionally comprises administering an additional agent, for example an additional active ingredient, e.g., a hair growth promoting agent. In some embodiments, the hair growth promoting agent is not minoxidil, finasteride, dutasteride, fluridil, a spironolactone, a cyproterone acetate, bicalutamide, flutamide, nilutamide, an inhibitor of an androgen receptor, an androgen antagonist, or an anti-androgen. In some embodiments, the hair growth promoting agent is minoxidil, finasteride, dutasteride, fluridil, a spironolactone, a cyproterone acetate, bicalutamide, flutamide, nilutamide, an inhibitor of an androgen receptor, an androgen antagonist, or an anti-androgen.

In some embodiments, at 3 months after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more vellus hair compared to immediately before the use of the methods described.

In some embodiments, at 3 months after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more vellus hair compared to immediately before the use of the methods described.

In some embodiments, at 3 months after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more non-vellus hair compared to immediately before the use of the methods described.

In some embodiments, at 3 months after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more non-vellus hair compared to immediately before the use of the methods described.

In some embodiments, at 3 months after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more terminal hair compared to immediately before the use of the methods described.

In some embodiments, at 3 months after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more terminal hair compared to immediately before the use of the methods described.

In some embodiments, at 3 months after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more intermediate hair compared to immediately before the use of the methods described.

In some embodiments, at 3 months after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more intermediate hair compared to immediately before the use of the methods described.

In some embodiments, at 3 months after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more vellus and non-vellus hair compared to immediately before the use of the methods described.

In some embodiments, at 3 months after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more more vellus and non-vellus or terminal hair compared to immediately before the use of the methods described.

In some embodiments, at 3 months after use of the methods described, ratio of non-vellus to vellus hair in the area of the scalp of the subject has increased by at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% compared to immediately before the use of the methods described.

In some embodiments, at 3 months after use of the methods described, ratio of non-vellus to vellus hair in the area of the scalp of the subject has increased by at least 5%, 10%, 15%, 20%, 25%, 30%, 35%, 40%, 45%, 50%, 55%, 60%, 65%, 70%, 75%, 80%, 85%, 90%, 95%, or at least 100% more more vellus and non-vellus or terminal hair compared to immediately before the use of the methods described.

In some embodiments, at 2 weeks after use of the methods described, the area of the

Attorney Docket No. 12718-064-999 scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more developing neofollicles compared to immediately before the use of the methods described.

In some embodiments, at 2 weeks after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more developing neofollicles compared to immediately before the use of the methods described.

In some embodiments, at 1, 2 or 3 weeks after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more developing neofollicles compared to immediately before the use of the methods described.

In some embodiments, at 1, 2, or 3 weeks after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more developing neofollicles compared to immediately before the use of the methods described. In some embodiments, at 1, 2 or 3 weeks after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more stimulated, activated, and reorganized follicular (e.g., branched) structures compared to immediately before the use of the methods described.

In some embodiments, at 1, 2, or 3 weeks after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more stimulated, activated, and reorganized follicular (e.g., branched) structures compared to immediately before the use of the methods described.

In some embodiments, at 3 months after use of the methods described, the area of the scalp of the subject has at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% more stimulated, activated, and reorganized follicular (e.g., branched) structures compared to immediately before the use of the methods described.

In some embodiments, at 3 months after use of the methods described, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more stimulated, activated, and reorganized follicular (e.g., branched) structures compared to immediately before the use of the methods described.

In some embodiments, at 3 months after the integumental perturbation, the area of the skin of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more hair growth or hair thickness compared to immediately before the integumental perturbation.

In addition, it is to be understood that the phraseology and terminology employed herein are for the purpose of description and should not be regarded as limiting. For example, the use of a singular term, such as, “a” is not intended as limiting of the number of items. Also the use of relational terms, such as but not limited to, “top,” “bottom,” “left,” “right,” “upper,” “lower,” “down,” “up,” “side,” are used in the description for clarity in specific reference to the Figures and are not intended to limit the scope of the invention or the appended claims. Further, it should be understood that any one of the features of the invention may be used separately or in combination with other features. Other systems, methods, features, and advantages of the invention will be or become apparent to one with skill in the art upon examination of the Figures and the detailed description. It is intended that all such additional systems, methods, features, and advantages be included within this description, be within the scope of the present invention, and be protected by the accompanying claims.

In certain embodiments, provided herein is a method for treating baldness or promoting hair growth, wherein the method comprises administration of valproic acid is administered as described in Section 6.1 and/or is formulated as described in Section 6.1.3 below. In a more specific embodiment, this method does not comprise integumental perturbation.

The written description are provided to teach any person skilled in the art to make and use the inventions for which patent protection is sought. The invention is capable of other embodiments and of being practiced and carried out in various ways. Those skilled in the art will appreciate that not all features of a commercial embodiment are shown for the sake of clarity and understanding. Persons of skill in the art will also appreciate that the development of an actual commercial embodiment incorporating aspects of the present inventions will require numerous implementation—specific decisions to achieve the developer's ultimate goal for the commercial embodiment. While these efforts may be complex and time-consuming, these efforts nevertheless would be a routine undertaking for those of skill in the art having the benefit of this disclosure.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

### 6.1 ADMINISTRATION OF VALPROIC ACID

In some embodiments, the methods described herein comprises administration (e.g., topical administration) of valproic acid (e.g., a pharmaceutical composition comprising valproic acid or a pharmaceutically acceptable salt, isotopic variant, or solvate thereof, that is intended to promote the growth of hair, including vellus hair, non-vellus hair, intermediate hair, terminal hair, increase hair thickness, prevent infection and/or promote healing, e.g., scarless healing, of the perturbed skin.

In some embodiments, the valproic acid or a pharmaceutically acceptable salt thereof is present in an amount of 0.5-30% wt % based on the total weight of the pharmaceutical composition. In some embodiments, the valproic acid or a pharmaceutically acceptable salt thereof is present in an amount of 2.0-25% wt % based on the total weight of the pharmaceutical composition. In some embodiments, the valproic acid or a pharmaceutically acceptable salt thereof is present in an amount of 0.5-1, 1-1.5, 1.5-2, 2-2.5, 2.5-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-21, 21-22, 22-23, 23-24, 24-25, or 25% wt % based on the total weight of the pharmaceutical composition. In some embodiments, the valproic acid or a pharmaceutically acceptable salt thereof is present at least in an amount of 0.5-1, 1-2, 2-3, 3-4, 4-5, 5-6, 6-7, 7-8, 8-9, 9-10, 10-15, 15-20, or 20% wt % based on the total weight of the pharmaceutical composition. In some embodiments, the valproic acid or a pharmaceutically acceptable salt thereof is present in an amount of 0.5, 1, 1.5, 2, 2.5, 3, 4, 5, 6, 7, 8, 9, 10, 11, 12, 13, 14, 15, 16, 17, 18, 19, 20, 21, 22, 23, 24, or 25% wt % based on the total weight of the pharmaceutical composition. In some embodiments, the valproic acid or a pharmaceutically acceptable salt thereof is present at least in an amount of 0.5, 1, 2, 3, 4, 5, 6, 7, 8, 9, 10, 15, or 20% wt % based on the total weight of the pharmaceutical composition. In some embodiments, the valproic acid or a pharmaceutically acceptable salt thereof is present at least in an amount 0.5% wt % based on the total weight of the pharmaceutical composition. In some embodiments, the valproic acid or a pharmaceutically acceptable salt thereof is present at least in an amount 1% wt % based on the total weight of the pharmaceutical composition. In some embodiments, the valproic acid or a pharmaceutically acceptable salt thereof is present at least in an amount 5% wt % based on the total weight of the pharmaceutical composition.

In some embodiments, a pharmaceutical composition is formulated for topical administration. In particular embodiments, the pharmaceutical composition formulated for topical administration is non-occlusive. In some embodiments, the non-occlusive pharmaceutical composition formulated for topical administration is an aqueous formulation (e.g., hydrogel), a non-aqueous formulation, an ointment, a suspension, or a cream (e.g., emulsion). In certain embodiments the wound healing gel is applied immediately after integumental perturbation and every day for about a week.

The methods described herein are suitable for achieving one, two, or more biological outcomes described in Section 6.4.1. Without being bound by theory, the methods described herein can induce hair follicle neogenesis, and can also stimulate, activate, and reorganize follicular structures in order to promote hair growth. Many conventional pharmacologic treatments for hair growth promotion (e.g., agents described in Section 6.5, below) encourage the switch from vellus to non-vellus or terminal hair. In a specific embodiment, the methods described herein are suitable for promoting the formation of stimulated, activated and reorganized hair follicle structures which correlate with increased vellus hair, if not non-vellus or terminal hair. By increasing the number of stimulated and activated hair follicles, and vellus hair or terminal hair, the methods described herein may be suitable for providing additional substrates for the action of these pharmacologic treatments. Thus, in certain aspects, methods described herein are suitable for increasing hair, increasing hair thickness, and/or yielding longer lasting hair. Accordingly, such treatments may be more effective, efficient, cost-effective, and user friendly as compared to the pharmacologic treatment alone. For example, fewer treatments may be required. The hair that results may be more cosmetically satisfactory, longer lasting, thicker, more uniform, longer, and properly pigmented hair.

For example, in certain aspects, methods described herein are suitable for yielding at a 0.25 to 0.5-fold, 0.5 to 1-fold, 1 to 1.25-fold, 1.25 to 1.5-fold, 1.5 to 2-fold, 2 to 2.5-fold, 2.5 to 3-fold, 3 to 3.5-fold, or 3.5 to 4-fold or more increase in one or more of the biological outcomes described in Section 6.4.1 as compared to treatment with the pharmacologic treatment alone. For example, in certain aspects, methods described herein are suitable for yielding a 0.25-fold, 0.5-fold, 1.25-fold, 1.5-fold, 2-fold, 2.5-fold, 3-fold, 3.5-fold, or 4-fold or more increase in one or more of the biological outcomes described in Section 6.4.1 as compared to treatment with the pharmacologic treatment alone. In one embodiment, a method of treatment described herein comprising integumental perturbation (e.g., by a needling device and/or adaptor described herein) and administration of a pharmacologic treatment (e.g., valproic acid or a pharmaceutically acceptable salt as described in Section 6.1.2 below) yields a 0.25 to 0.5-fold, 0.5 to 1-fold, 1 to 1.25-fold, 1.25 to 1.5-fold, 1.5 to 2-fold, 2 to 2.5-fold, 2.5 to 3-fold, 3 to 3.5-fold, or 3.5 to 4-fold or more increase in one or more of the biological outcomes described in Section 6.4.1 as compared to treatment with valproic acid or a pharmaceutical acceptable salt alone (i.e., without the integumental perturbation step). For example, in a specific embodiment, a method of treatment described herein comprising integumental perturbation (e.g., by a needling device and/or adaptor described herein) and administration of a pharmacologic treatment (e.g., valproic acid or a pharmaceutically acceptable salt as described in Section 6.1.2 below) yields a 0.25-fold, 0.5-fold, 1-fold, 1.25-fold, 1.5-fold, 2-fold, 2.5-fold, 3-fold, 3.5-fold, or 4-fold or more increase in one or more of the biological outcomes described in Section 6.4.1 as compared to treatment with valproic acid or a pharmaceutical acceptable salt alone (i.e., without the integumental perturbation step). In a specific embodiment, the methods described herein are suitable for yielding one or more of the biological outcomes described in Section 6.4.1 in at least 1 to 5%, 5 to 10%, 10 to 20%, 20 to 30%, 30 to 40%, 40 to 50%, 50 to 60%, 60 to 70%, 70 to 80%, 80 to 90%, or 90 to 98% or more of the time that a control method requires to achieve the one or more biological outcomes. In a specific embodiment, the methods described herein are suitable for yielding one or more of the biological outcomes described in Section 6.4.1 in at least 5%, 10%, 20%, 30%, 40%, 50%, 60%, 70%, 80%, 90%, or 98% of the time that a control method requires to achieve the one or more biological outcomes. In one embodiment, the methods described herein are suitable for yielding one or more of the biological outcomes described in Section 6.4.1 in at most 1 to 5%, 5 to 10%, 10 to 20%, 20 to 30%, 30 to 40%, 40 to 50%, 50 to 60%, 60 to 70%, 70 to 80%, 80 to 90%, or 90 to 98% of the time that a control method requires to achieve the one or more biological outcomes. In a specific embodiment, the methods described herein are suitable for yielding one or more of the biological outcomes described in Section 6.4.1 in at most 5%, 10%, 20%, 30%, 40%, 50%, 60%, 70%, 80%, 90%, or 98% of the time that a control method requires to achieve the one or more biological outcomes. In a specific embodiment, the control method is a method comprising microdermabrasion and administration of valproic acid or a pharmaceutical acceptable salt (e.g., valproic acid or a pharmaceutical acceptable salt as described in Section 6.1.2, below). The synergistic effect of the integumental perturbation and administration of valproic acid or a pharmaceutical acceptable salt as described herein may be measured as an improvement over a control subject (or a control skin site on the same subject) receiving valproic acid or a pharmaceutical acceptable salt and not the integumental perturbation.

### 6.1.1 Doses

The dose of the valproic acid or pharmaceutically acceptable salt used in accordance with the methods provided herein should be adjusted so that maximum benefit is achieved while reducing potential side effects.

In some embodiments, the target concentration of the valproic acid or pharmaceutically acceptable salt should be maintained in the skin or blood, and preferably the skin, for at least 1 day; at least 2 days; at least 3 days; at least 4 days; at least 5 days; at least 6 days; at least 7 days; at least 8 days; at least 9 days; at least 10 days; at least 11 days; at least 12 days; at least 13 days; at least 14 days; at least 15 days; at least 16 days; at least 19 days; or at least 21 days; and, in certain embodiments, not more than 28 days. In certain embodiments, the target concentration of the valproic acid or pharmaceutically acceptable salt is maintained in skin or blood, and preferably the skin, for 1 month or more, 2 months or more, 3 months or more, 3 to 6 months or more, or 6 to 12 months or more. This can be accomplished using, e.g., repeated applications of the valproic acid or pharmaceutically acceptable salt or a single application of a sustained release or extended release agent formulation. For example, a modified release form can be used to achieve the target concentration of the valproic acid or pharmaceutically acceptable salt for shorter maintenance periods (i.e., for at least 1, 2 or 3 days). Maintenance periods longer than 3 days may require repeated application of the agent treatments. In some embodiments, it is preferable to allow the concentration of the valproic acid or pharmaceutically acceptable salt to decline between dosages.

In some embodiments, valproic acid or a pharmaceutically acceptable salt thereof (e.g., a pharmaceutical composition comprising valproic acid or a pharmaceutically acceptable salt thereof) is administered for 1, 2, 3 months or more. In some embodiments, the valproic acid or a pharmaceutically acceptable salt thereof is administered for over 3 months.

Care should be taken to avoid toxicity. In this regard, a dosage should be chosen that maximizes efficacy while minimizing toxicity. Patients should be monitored for toxic side effects according to standard clinical practice. In some embodiments, valproic acid or pharmaceutically acceptable salt doses should be adjusted on the basis of the blood concentration (serum or plasma) drawn (by convention) 12 or 24 hours after the last dose of the agent. It may be possible to predict dosage requirements for an individual patient based on the results of administration of a single test dose, followed by a skin and/or blood sample assay (plasma or serum) at the peak concentration time; followed by blood sample assays to monitor toxicity at the 12 hour or 24 hour trough concentration; and 24 or 48 or 96 hours later (when hair growth-promoting agent is generally eliminated) which serves as the control value. Once the dose is established for a patient, routine monitoring for toxicity is recommended.

In another embodiment, the dose of the valproic acid or pharmaceutically acceptable salt is a dose sufficient to achieve one or more of the biological outcomes described in Section 6.4.1.

### 6.1.2 Valproic Acid

Described herein are methods of promoting hair growth by subjecting an area of the skin of a subject to integumental perturbation and administration of valproic acid or a pharmaceutically acceptable salt, isotopic variant, or solvate thereof, e.g., a pharmaceutical composition comprising valproic acid or a pharmaceutically acceptable salt, isotopic variant, or solvate thereof. Valproic acid can be derived from valeric acid or valerian. In some embodiments, the methods provided comprise administration of valerian, valeric acid, or an extract, product, or derivative thereof (e.g., valproic acid).

Valproic acid is also referred to as 2-propylpentanoic acid, dipropylacetic acid, dipropyl acetate, calcium valproate, semisodium valproate, sodium valproate, valproate, magnesium valproate, convulsofin, ergenyl, propylisopropylacetic acid, depakote, divalproex, divalproex sodium, and vupral. Valproic acid has also been referred to as absenor, convulex, depakene, depakine, deprakine, depalept, encorate, epival, epilim, stavzor, valcote, and valpakine. Valproic acid can be depicted by the following structure:

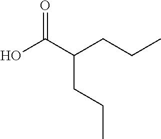

Valproic acid has a molecular weight of 144.214 g/mol.

### 6.1.3 Pharmaceutical Formulations

The pharmaceutical compositions described herein comprise valproic acid or a pharmaceutically acceptable salt, isotopic variant, or solvate as described in Section 6.1.2 and a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. The pharmaceutical compositions described herein comprise a pharmaceutically acceptable carrier (also referred to as a pharmaceutically acceptable excipients), i.e., a pharmaceutically-acceptable material, composition, or vehicle, such as a liquid or solid filler, diluent, solvent, an encapsulating material, or a complexation agent. In one embodiment, each component is “pharmaceutically acceptable” in the sense of being chemically compatible with the other ingredients of a pharmaceutical formulation, and biocompatible, when in contact with the biological tissues or organs of humans and animals without excessive toxicity, irritation, allergic response, immunogenicity, or other problems or complications, commensurate with a reasonable benefit/risk ratio. See, Remington: The Science and Practice of Pharmacy, 2005, 21st ed., Philadelphia, Pa.: Lippincott Williams & Wilkins; Rowe et al., eds., 2005, Handbook of Pharmaceutical Excipients, 5th ed., The Pharmaceutical Press and the American Pharmaceutical Association; Ash & Ash eds., 2007, Handbook of Pharmaceutical Additives, 3rd ed., Gower Publishing Company; Gibson ed., 2009, Pharmaceutical Preformulation and Formulation, 2nd ed., Boca Raton, Fla.: CRC Press LLC, each of which is incorporated herein by reference.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, forms for topical administration such as a cream may contain excipients not suited for use in transdermal or intravenous administration. The suitability of a particular excipient depends on the specific active ingredients in the dosage form. Exemplary, non-limiting, pharmaceutically acceptable carriers for use in the valproic acid formulations described herein are the cosmetically acceptable vehicles provided in International Patent Application Publication No. WO 2005/120451, which is incorporated herein by reference in its entirety.

The pharmaceutical compositions disclosed herein may be formulated to include an appropriate aqueous vehicle, including, but not limited to, water, saline, physiological saline or buffered saline (e.g., phosphate buffered saline (PBS)), sodium chloride for injection, Ringers for injection, isotonic dextrose for injection, sterile water for injection, dextrose lactated Ringers for injection, sodium bicarbonate, or albumin for injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, lanolin oil, lanolin alcohol, linoleic acid, linolenic acid and palm seed oil. Suitable water-miscible vehicles include, but are not limited to, ethanol, wool alcohol, 1,3-butanediol, liquid polyethylene glycol (e.g., polyethylene glycol 300 and polyethylene glycol 400), propylene glycol, glycerin, N-methyl-2-pyrrolidone (NMP), N,N-dimethylacetamide (DMA), and dimethyl sulfoxide (DMSO). In one embodiment, the water-miscible vehicle is not DMSO.

Suitable isotonic agents include, but are not limited to, sodium chloride, glycerin, and dextrose. Suitable buffering agents include, but are not limited to, phosphate, glutamate and citrate. Suitable suspending and dispersing agents include but are not limited to sodium carboxymethylcelluose (CMC), hydroxypropyl methylcellulose (HPMC), polyvinyl alcohol (PVA), and polyvinylpyrrolidone (PVP). Suitable emulsifying agents include but are not limited to, including polyoxyethylene sorbitan monolaurate, polyoxyethylene sorbitan monooleate 80, and triethanolamine oleate. Suitable sequestering or chelating agents include, but are not limited to, EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

A product for application to the scalp or face may additionally be formulated so that it has easy rinsing, minimal skin/eye irritation, no damage to existing hair, has a thick and/or creamy feel, pleasant fragrance, low toxicity, good biodegradability, and a slightly acidic pH (pH less than 7), since a basic environment weakens the hair by breaking the disulfide bonds in hair keratin.

Provided herein are pharmaceutical compositions for administration to skin following (and optionally before or during) integumental perturbation. In certain embodiments, the pharmaceutical composition is formulated for topical administration to skin. In a particular embodiment, the pharmaceutical composition is administered to an area of the skin that will be, is being, or that has been subjected to integumental perturbation in accordance with a method described herein. In some embodiments, the pharmaceutical composition is administered with a non-occlusive wound covering. In some such embodiments, a pharmaceutical composition is administered in order to heal the integumentally perturbed skin by primary intention. In some such embodiments, a pharmaceutical composition is administered in order to heal the integumentally perturbed skin by secondary intention. In some such embodiments, a pharmaceutical composition is administered in order to heal the integumentally perturbed skin by tertiary intention. In some such embodiments, a pharmaceutical composition is administered in order to heal the integumentally perturbed skin more slowly than usually indicated for that kind of wound. This may enhance scarless wound healing and/or prolong the period during which hair growth in the wounded area of skin is promoted. In some such embodiments, a pharmaceutical composition promotes wound healing with no or minimal scarring.

In non-limiting embodiments, a pharmaceutical composition for treatment is formulated for topical administration as a gel, hydrogel, emulsion, solution, suspension, cream, ointment, dusting powder, dressing, elixir, lotion, suspension, tincture, paste, powder, crystal, foams film, aerosol, irrigation, spray, suppository, stick, bar, ointment, bandage, wound dressing, microdermabrasion or dermabrasion particle, drop, transdermal patch, or dermal patch. In particular embodiments, the pharmaceutical composition is an aqueous formulation (e.g., hydrogel), a non-aqueous formulation, ointment, or cream (e.g., emulsion). In one embodiment, the composition is a hydrogel. In some embodiments, the composition is occlusive. In other embodiments, the composition is non-occlusive. The compositions may be administered via any topical means of delivery known in the art. In particular embodiments, the composition is administered using a drug delivery system, such as described in Section 6.1.4 infra.

In some embodiments, the pharmaceutical composition for treatment contains an active ingredient or active ingredients, such as described in Section 6.1.2 below.

In some embodiments, the formulation of the pharmaceutical composition for treatment is varied in order to control the rate of release of active ingredients (where present) in the composition. This may be accomplished by, for example, varying the molecular fluidity of the carrier, without changing its hydrophobicity, such as by varying the petrolatum to mineral oil ratio. In one embodiment, the pharmaceutical formulation is an ointment, comprising petrolatum, mineral oil, and lanolin alcohol. Exemplary formulations prepared in accordance with such embodiments are provided in the Examples below. In another embodiment, release of active ingredients can be modulated by varying the hydrophobic/ hydrophilic ratio of the formulation, for example, by preparing a petrolatum/water emulsion. Exemplary formulations prepared in accordance with such embodiments are provided in the Examples below.

### (I) Hydrogels

In one embodiment, the pharmaceutical composition for administration of valproic acid is formulated as an aqueous hydrogel. In one embodiment, the aqueous hydrogel comprises, in addition to valproic acid, Carbopol 980, methyl paraben, propyl paraben, propylene glycol, glycerine, and water. In one embodiment, a hydrogel formulation comprises citric acid, CMC, methyl paraben, propyl paraben, allantoin, alginate, and water. Exemplary formulations prepared in accordance with such embodiments are provided in the Examples below. In one embodiment, a hydrogel has the following composition: glycerol, carboxymethyl cellulose, allantoin, sodium alginate, methyl paraben, propyl paraben, water (Q.S.), and sodium hydroxide (pH adjusted to 6.5-7.5). Methods for formulating hydrogels are described in detail in the Examples below. These methods may be adapted to generate other hydrogel formulations using methods known in the art and described herein.

In certain embodiments, a hydrogel contains approximately 75%, 80%, 85%, 90%, or 95% water. In a particular embodiment, the hydrogel contains 90% water. Preferably, the hydrogel has one or more or all of the following characteristics: is transparent, odorless, colorless, has a viscosity (at 25° C.) of, e.g., 2,000-10,000 cP, 2,000-8,000 cP, or 6,000-10,000 cP (measured using, for example, a rheometer), has assay and dose uniformity (which can be measured by, e.g., flame photometry or atomic adsorption spectrometry (AAS)), has an emollient “smooth-feel” texture, could be easily applied to skin, readily spreads over a surface, has minimal migration to surrounding sites, has minimal run off, has a neutral pH (e.g., pH 6.5-7.5), is sterile, is stable for an extended period (e.g., 1 week or more, 2 weeks or more, 4 weeks or more, 8 weeks or more, 12 weeks or more, 4 months or more, 6 months or more, 1 year or more, or 2 years or more) at one or more temperature conditions (e.g., 4 ° C., 25 ° C. and 40 ° C.) with respect to, for example, strength, viscosity, and homogeneity. In one embodiment, the hydrogel is stable at room temperature for up to 4 weeks or more. In one embodiment, the hydrogel is stable at room temperature for up to 8 weeks or more. In one embodiment, the hydrogel is stable at 4 ° C. for up to 6 months or more. In one embodiment, the hydrogel is stable at 4 ° C. for up to 1 year or more. In certain embodiments, a hydrogel is prepared with the excipients and an amount of active ingredient chosen to contribute to one or more of the foregoing or following attributes, which may be desirable for a topical formulation for use in the methods described herein: viscosity (e.g., imparted by carboxymethyl cellulose), surface wetting ability and prevention of “dry-out” (e.g., imparted by glycerol), preservative effectiveness (e.g., imparted by parabens, such as methyl or propyl parabens, although in certain embodiments, a paraben-free formulation may also be generated), maintenance of pH, stability (e.g., imparted by altering the strength of surfactants used in the hydrogel) and pharmacokinetic properties (such as rate of release of active ingredient from the formulation, and peak and trough concentrations of active ingredient in skin and blood). In embodiments where the formulation is for administration to skin that is wounded or that may be wounded, excipients that are wound compatible, contribute to sterility, wound healing, and/or aid in cell attachment and/or proliferation may be included, such as, e.g., allantoin or sodium alginate.

In some embodiments, the hydrogel is formulated so that it releases active ingredients, where present, at varying rates. In some embodiments, most or all of the active ingredient is released from the formulation within 2 hours, within 4 hours, within 8 hours, within 10 hours, within 12 hours, within 16 hours, within 24 hours, within 36 hours, within 48 hours, within 3 days, within 5 days, within 7 days, within 10 days, within 14 days, within 30 days, or within 2 months or more. In a specific embodiment, most or all of any active ingredient is released from a hydrogel described herein within 12 hours. In one embodiment, all of the active ingredient is released from the hydrogel within 12 hours. In another embodiment, most or all of the active ingredient is released from a hydrogel described herein within 24 hours. In one embodiment, the formulation is an “immediate release” formulation, i.e., releases 90-100% of active ingredient within the first day of administration. In another embodiment, the formulation is an “Intermediate Release” formulation, i.e., releases 90-100% of active ingredient within 1 to 3 days of administration. In another embodiment, the formulation is a “Sustained Release” formulation, i.e., releases 90-100% of active ingredient within 3 to 7 days of administration.

### (II) Creams

In another particular embodiment, the pharmaceutical composition formulated for topical administration of valproic acid is in the form of an emulsion, e.g., a cream. In one embodiment, the cream is an oil/water emulsion.

In certain embodiments, a cream contains approximately 75%, 80%, 85%, 90%, or 95% water. In certain embodiments, the cream (e.g., dispersion, suspension, colloid or emulsion) has one or more or all of the following characteristics: is odorless, colorless upon application to the skin, has a viscosity (at 25° C.) of, e.g., 2,000-10,000 cP, 2,000-8,000 cP, or 6,000-10,000 cP (measured using, for example, a rheometer), has assay and dose uniformity (which can be measured by, e.g., flame photometry or atomic adsorption spectrometry (AAS)), has an emollient “smooth-feel” texture, could be easily applied to skin, readily spreads over a surface, has minimal migration to surrounding sites, has minimal run off, has a neutral pH (e.g., pH 6.5-7.5), is sterile, is stable for an extended period (e.g., 1 week or more, 2 weeks or more, 4 weeks or more, 8 weeks or more, 12 weeks or more, 4 months or more, 6 months or more, 1 year or more, or 2 years or more) at one or more temperature conditions (e.g., 4° C., 25° C. and 40° C.) with respect to, for example, strength, viscosity, and homogeneity. In one embodiment, the cream is stable at room temperature for up to 4 weeks or more. In one embodiment, the cream is stable at room temperature for up to 8 weeks or more. In one embodiment, the cream is stable at 4° C. for up to 6 months or more. In one embodiment, the cream is stable at 4° C. for up to 1 year or more. In certain embodiments, a cream is prepared with the excipients and an amount of active ingredient chosen to contribute to one or more of the foregoing or following attributes, which may be desirable for a topical formulation for use in the methods described herein: viscosity, surface wetting ability and prevention of “dry-out,” preservative effectiveness, maintenance of pH, stability (e.g., imparted by altering the strength of surfactants used in the cream), and pharmacokinetic properties (such as rate of release of any active ingredients from the formulation, and peak and trough concentrations in skin and blood). In embodiments where the formulation is for administration to skin that is wounded or that may be wounded, excipients that are wound compatible, contribute to wound healing, and/or aid in cell attachment and/or proliferation may be included, such as, e.g., allantoin or sodium alginate.

The rate of release of active ingredients, for example valproic acid or a pharmaceutically acceptable salt thereof, from the cream may be modified by one or more of the following: incorporating the formulation into different scaffolds, modifying the concentration of valproic acid or a pharmaceutically acceptable salt thereof in the formulation, or modifying the types and concentrations of excipients. For example, in one embodiment, the rate of release of active ingredients from the cream may be decreased by decreasing the concentration of hydrophilic polymers in the cream. In some embodiments, the rate of release of active ingredients from the cream may be altered by varying the concentration of cetearyl alcohol, lanolin alcohol, or by varying the types of aqueous or non-aqueous carrier(s), and preferably non-aqueous carrier(s) (e.g., silicone, mineral oil, petrolatum, etc.), used.

In some embodiments, most or all of the active ingredient is released from the formulation within 2 hours, within 4 hours, within 8 hours, within 10 hours, within 12 hours, within 16 hours, within 24 hours, within 36 hours, within 48 hours, within 3 days, within 5 days, within 7 days, within 10 days, within 14 days, within 30 days, or within 2 months or more. In a specific embodiment, most or all of the active ingredient is released from a cream described herein within 10 hours. In one embodiment, all of the active ingredient is released from the cream within 10 hours. In another embodiment, most or all of the active ingredient is released from a cream described herein within 24 hours. In one embodiment, the formulation is an “immediate release” formulation, i.e., releases 90-100% of active ingredient within the first day of administration. In another embodiment, the formulation is an “Intermediate Release” formulation, i.e., releases 90-100% of active ingredient within 1 to 3 days of administration. In another embodiment, the formulation is a “Sustained Release” formulation, i.e., releases 90-100% of active ingredient within 3 to 7 days of administration.

In a specific embodiment, the cream is an immediate release formulation. Such a formulation may be generated using a two-phase system: (i) an aqueous phase for dissolving any active ingredients and hydrophilic excipients and (ii) a non-aqueous phase for dissolving hydrophobic polymers. In an exemplary embodiment, the cream is a water-in-oil emulsion, which acts not only act as a biocompatible skin emollient, but also as a delivery system for any active ingredients.

In another embodiment, the cream is an intermediate release formulation. In one embodiment, the intermediate release cream formulation is an emulsion prepared by homogenization of two phases, as described, e.g., for the immediate release cream formulation above.

In another embodiment, the cream is a sustained release formulation. In one embodiment, the sustained release cream formulation is prepared by homogenization of two phases (an aqueous phase and a non-aqueous phase), as described, e.g., for the immediate and intermediate release cream formulations above, but by decreasing the concentration of hydrophilic polymers in the non-aqueous phase.

The foregoing formulations for topical administration may be administered in accordance with any embodiments described herein. For example, in specific embodiments, a 50 kg patient is administered a single droplet of a hydrogel described herein at 3 sites, twice daily. In some embodiments, the hydrogel is administered once daily. In some embodiments, the hydrogel is administered twice daily. In some embodiments of a twice daily treatment regimen, doses are administered 6 hours apart, or 7 hours apart, or 8 hours apart, or 9 hours apart, or 10 hours apart, or 11 hours apart, or 12 hours apart. In a particular embodiment, the doses are administered 7 to 8 hours apart.

### 6.1.4 Applicator Devices

The methods described, comprising administration of valproic acid or a pharmaceutically acceptable salt, isotopic variant, or solvate thereof, can be performed using applicator devices (e.g., fluid or drug applicators). The fluid or drug applicators described herein may be used with any type of fluid. In an example, the fluid applicator is a drug applicator that is used for the application of valproic acid or a pharmaceutically acceptable salt thereof to a subject's scalp or face or for a variety of different applications using different chemicals. With respect to all embodiments of the drug or fluid applicators, it should be appreciated that any type of fluid may be used, and that this invention is not limited to the use of a drug. For convenience, the devices may be referred to throughout the specification as “drug applicators”, “fluid applicators”, or simply “applicators”. Methods of using the applicator devices described in this application are provided in more detail in Section (ii), below.

In a specific embodiment, other compounds that may be used with the applicator include agents described in Section 6.5, below. In a specific embodiment, the compound(s) that may be used with the applicator is in a dose as described in Section 6.1.1, below.

In a specific embodiment, the integumental perturbation performed by the needling devices and needling adaptors described herein is followed by the application of a compound to the skin.

### (I) Aspects of the Described Embodiments of the Applicator Device

In all embodiments of the drug applicators described above, the drug applicators may be programmed remotely using an external or mobile device via Bluetooth® or other wireless communications. In an example, the total dose is 1 ml per treatment and is delivered in five pulses of 0.2 ml each (or an appropriate number of smaller pulses). Each pulse is a full-stroke dispenser actuation. In an example, the drug applicators may be programmed to perform a Massage +Dispense cycle for 1 minute that is followed by a Massage-only cycle for 5 minutes. A number of different programmable cycles may also be selected by a user.

It should be appreciated that a number of different compounds, liquids or drugs may be used with the drug applicators and cartridges, and cartridges may be sold separately including a variety of different compounds, liquids or drugs. For example, in an embodiment, a cartridge contains multiple compounds for simultaneous delivery. In addition to valproic acid or a pharmaceutically acceptable salt thereof, as described above in Section 6.1.2, a number of different drugs or compounds may be used including proteasome inhibitor such as lactacystin, a peptidyl aldehyde, or pentoxyfilline (PTX), and the active ingredients described in Section 6.5, below.

For example, water cartridges may be used for practicing or a cartridge including a healing solution may be used by those who have had micro-needling procedures. Further, a special cleaning cartridge can be provided for cleaning the applicator systems, among other types of fluid and viscous material cartridges. Alternative design embodiments for the cartridges may include smaller sized cartridges providing for daily or unit dosing such as with metered dosing for syringes.

In an aspect, it should be appreciated that advantages of the applicators include massaging and parting hair simultaneously while dispensing a chemical, omni-directional movement of the massage heads, a charging station acting as a light pipe and luminous display, buttonless powering, remote control and smart phone application control and monitoring, among other features. The applicators deliver drug directly to the skin and the massage mode spreads the drug over the skin providing for more consistent and evenly distributed administration of the drug. Among other advantages, the described drug applicators provide a more effective means of applying pharmaceutical compositions (e.g., pharmaceutical composition comprising valproic acid or a pharmaceutically acceptable salt thereof) to a subject's scalp.

In another aspect, it should be appreciated that the cartridges described in all embodiments above may include a two-chamber or multi-chamber cartridge for housing and dispensing two or more drugs separately, or mixing two or more drugs and dispensing the two or more drugs together. In another another aspect, the cartridges described in all embodiments above may include a single chamber cartridge for housing and dispensing multiple drugs (i.e., more than one drug) simultaneously. Further, all embodiments of the applicators described above include an applicator that is capable of detecting information related to an inserted cartridge by detecting labeling provided on the inserted cartridge using RFID technology.

### (II) Methods of Using the Applicator Device

In an example, methods of using applicator devices include providing an applicator comprising having a housing, a drug delivery cartridge carried by the housing, and a massage head which is mounted on the housing, powering on the applicator; and dispense a drug automatically by a dispensing mechanism that is linked to movement of the massage head or by any other dispensing mechanism. In an example, the applicator devices are programmed by a physician or any user to follow a preset cycle of massage-only or massage and dispense; for example, a 5-minute massage cycle is followed by a 1-minute massage and dispense cycle. The device may be programmed from a keypad on the device or wirelessly using an external device.

The method may further include removing the massage head and attached drug delivery cartridge; and replacing the massage head and attached drug delivery cartridge with another massage head having another drug delivery cartridge and other massage head nodules having a different nodule arrangement. The method may further include receiving notifications from and interacting with the applicator using a remote user interface.

In a specific embodiment, the applicator device is used in a method of treatment described in Section 6.3.1.

### 6.2 INTEGUMENTAL PERTURBATION

Methods described herein comprise integumental perturbation and administration of valproic acid or a pharmaceutically acceptable salt thereof. In one embodiment, integumental perturbation causes only superficial wounding to the area of skin on which hair growth is desired. In a particular embodiment, the extent of wounding is minimized by controlling the depth of integumental perturbation. For example, integumental perturbation can be controlled to limit perturbation to part or all of the epidermis, to part or all of the stratum corneum, or deeper into the papillary dermis, reticular dermis, and/or hypodermis. The occurrence of pinpoint bleeding would indicate disruption of the stratum corneum, epidermis (or part thereof) and portions of the upper layer of the dermis, such as the superficial papillary dermis. The occurrence of increased bleeding would indicate deeper penetration into (and thus disruption of) the deeper papillary dermis and reticular dermis layer.

In one embodiment, integumental perturbation does not remove the epidermis. In some embodiments, integumental perturbation achieves removal of part of the epidermis. In some embodiments, integumental perturbation removes the entire epidermis. In some embodiments, integumental perturbation removes all of the epidermis and part of the dermis. In some embodiments, integumental perturbation removes part of the stratum corneum. In some embodiments, integumental perturbation removes the stratum corneum. In some embodiments, integumental perturbation removes part of the papillary dermis. In some embodiments, integumental perturbation removes part of the more superficial papillary dermis. In some embodiments, integumental perturbation removes part of the deeper papillary dermis. In some embodiments, integumental perturbation removes the papillary dermis. In some embodiments, integumental perturbation removes the reticular dermis, or part of the reticular dermis. The depth of integumental perturbation depends on the thickness of the skin at a particular treatment area. For example, the skin of the eyelid is significantly thinner than that of the scalp. The occurrence of pinpoint bleeding indicates that the epidermis and portions of the dermis have been removed. Deeper penetration can result in much more bleeding, and the perturbation can go as deep as the hypodermis.

In particular embodiments, integumental perturbation is done to a clinical endpoint of pinpoint bleeding. In some embodiments, the depth reaches the level of blood vessels of the follicular papilla. In some embodiments, the depth does not go deeper than the level of blood vessels of the capillary loops in the dermal papilla, e.g., the area of papillary dermis in between rete pegs. In some embodiments, integumental perturbation does not penetrate the dermis. In some embodiments, integumental perturbation does not completely remove all, or in some embodiments, most, of the hair follicles in an area of treated skin. In one embodiment, integumental perturbation does not penetrate the reticular dermis. In one embodiment, integumental perturbation does not penetrate more than halfway through the papillary dermis.

In some embodiments, integumental perturbation penetrates the skin to a depth of between 500 and 2500 μm, 500 and 2300 μm, 500 and 2000 μm, 500 and 1500 μm, 500 and 1300 μm, 500 and 1500 μm, 500 and 1100 μm, 500 and 1000 μm, or 500 and 750 μm.

In some embodiments, the integumental perturbation penetrates the skin to a depth of 500 μm to 2.5 mm. In some embodiments, the integumental perturbation is performed by dermabrasion, laser, or controlled integumental perturbation. In some embodiments, the integumental perturbation is performed by dermabrasion. In some embodiments, the integumental perturbation is performed until pinpoint bleeding occurs. In some embodiments, the integumental perturbation is performed by a needling device or drug applicator device.

In some embodiments, the maximum depth of the integumental perturbation is 500, 600, 700, 800, 900, 1000, 1100, 1200, 1300, 1400, 1500, 1600, 1700, 1800, 1900, 2000, 2100, 2200, 2300, 2400 or 2500 μm.

In some embodiments, integumental perturbation penetrates the skin to a depth of at least 500, 600, 700, 800, 900, 1000, 1100, 1200, 1300, 1400, 1500, 1600, 1700, 1800, 1900, 2000, 2100, 2200, 2300, 2400 or 2500 μm.

In some embodiments, integumental perturbation is performed using micro-needling (see Section 6.2.1) wherein the microneedles penetrate the skin to a depth of approximately 500, 600, 700, 800, 900, 1000, 1100, 1200, 1300, 1400, 1500, 1600, 1700, 1800, 1900, 2000, 2100, 2200, 2300, 2400 or 2500 μm. In a specific embodiment, the microneedles penetrate the skin to a depth of approximately 700 to 900, 750 to 850, or approximately 800 μm.

In a specific embodiment, integumental perturbation removes the first 10-30 μm of these dead skin cells.

In a specific embodiment, integumental perturbation removes the stratum corneum and part or all of the epidermis by removing the first 30-100 μm of the skin. This is not deep enough to remove the sebaceous gland, bulge, or hair papilla of existing follicle structures. The removal of the epidermis can be detected by the appearance of a shiny, smooth, whiteish layer of skin.

In a specific embodiment, integumental perturbation penetrates at a depth of 0.8 mm (e.g., roughly at level of arrector pili muscle and sebaceous gland in an intact pre-existing follicle).

In a specific embodiment, integumental perturbation removes the stratum corneum, all of the epidermis, and disruption of the papillary dermis (e.g., between 100 μm and 150 μm of the skin). Disruption of the papillary dermis can be detected by the appearance of small pinpoints of blood in the treated area.

In a specific embodiment, integumental perturbation removes the stratum corneum, the full epidermis, and part of the dermis down to approximately 200 um.

### 6.2.1 Needling Devices

In a specific embodiment, a needling device and/or a needling adaptor are suitable for performing integumental perturbation on a subject. Advantages of the needling devices described in this application include protecting a reusable main unit or needling device having a removable sheath or adaptor to provide ease of use by a patient or physician in performing needling operations for a number of different procedures.

In certain aspects, the needling device and/or adaptor is used in a fashion that exerts control over the extent of integumental perturbation (e.g., targeted cutaneous perturbation (TCP)) and/or control over the way in which the integumentally perturbed skin heals.

### (I) Aspects of the Described Embodiments of the Needling Device

In an aspect, the needling devices and needling adaptors described in all embodiments above suitable to promote hair follicle neogenesis through targeted cutaneous perturbation (TCP) of the skin (e.g., the scalp). In a specific embodiment, TCP refers to integumental perturbation of one or more epidermal layers, for example, the basal and/or suprabasal epidermal layers. In a specific embodiment, the integumental perturbation performed by the needling devices and needling adaptors described herein is followed by the application of a compound to the skin. Without being bound by any particular theory, TCP triggers generation of new hair follicles by induction of epithelial stem cells. See, e.g., Section 6.2.1 for a more detailed discussion of integumental perturbation and methods of using the needling devices and needling adaptors. In an example, the needling devices and needling adaptors of all embodiments may form a combined rechargeable, battery powered, handheld instrument. The devices are used to perform TCP by reciprocating a needle array into and out of the scalp to “injure” the scalp. The hand piece assemblies include a durable and reusable needling device and a single-use needling adaptor that also acts as a protective barrier such that the needling device is not exposed to blood or other contaminated materials.

In an example, the needling devices of all embodiments may include a sensor for detecting whether the needling adaptor is a previously used needling adaptor so as to disallow reuse of the previously used needling adaptor. The needling device and/or the needling adaptor may also include a controller for determining when the device is powered off and automatically retracting the needles and/or adjusting the needle penetration depth to the most retracted position in response to the device being powered off. The needling device may also be used in combination with guides that fit over the needle array in order to direct movement through a subject's hair; for example, the guides can be similar to hair clipper attachments. This limits the lateral movement of the needling device and guides a user's hand in moving using straight line strokes.

The adaptors of all embodiments, being a different component of the combined device, provide complete insulation of the needling device from the needles of the needling head. That is, because the adaptor is a replaceable, removable, and disposable sheath, this prevents blood contamination of the needling device and prevents cross-contamination between different users of the needling device. Equally important, these devices reduce the amount of time needed to begin a needling operation because a doctor receives a sheath or adaptor that is pre-sterilized once removed from its packaging and the sheath protects the main needling device unit from any contamination, so that it may be used immediately after with another sheath or adaptor. This function allows for safe and easy use of the devices.

In an example, the speed of needling may include three discrete speeds (80 Hz, 100 Hz, 120 Hz), and may range from 60 Hz to 140 Hz. The speed of needling includes at least 60 Hz, at least 70 Hz, at least 80 Hz, at least 90 Hz, at least 100 Hz, at least 110 Hz, at least 120 Hz, at least 130 Hz, at most 70 Hz, at most 80 Hz, at most 90 Hz, at most 100 Hz, at most 110 Hz, at most 120 Hz, at most 130 Hz, and at most 140 Hz. In addition, needle penetration depth adjustment may include incremental needle penetration depth adjustment (0.5 mm above skin surface—2.5 mm below skin surface). This prevents the dragging of the needle within the subject's skin so that if the needles are in the 0.5 mm retracted position above the skin, the needles are fully retracted and do not drag within the subject's skin while the device is being moved by the user. Reducing drag and having a light source lighting the target region provides for higher quality procedure and more precise, more consistent needling.

With regards to penetration depth control of the needles, a variety of different penetration depth adjustments may be available. In an embodiment, penetration depth may range from 0.5 millimeters in the retracted position to 2.5 millimeters; however, it should be appreciated that the penetration depth of the needles is available in a broader range. For example, the penetration depth may include a range from −0.5 millimeters where the needles are in a completely retracted position to a range of 5 millimeters or more. The penetration depth may include at least −0.5 millimeters, at least 0 millimeters, at least 0.5 millimeters, at least 1 millimeter, at least 1.5 millimeters, at least 2 millimeters, at least 2.5 millimeters, at least 3 millimeters, at least 3.5 millimeters, at least 4 millimeters, at least 4.5 millimeters, at most 0.5 millimeters, at most 1 millimeter, at most 1.5 millimeters, at most 2 millimeters, at most 2.5 millimeters, at most 3 millimeters, at most 3.5 millimeters, at most 4 millimeters, at most 4.5 millimeters, or at most 5 millimeters.

In one aspect, a needling device or needling adaptor described herein is suitable for disrupting skin to a penetration depth of between 500 μm to 2500 μm (e.g., to a maximum depth of 500, 1000, 1100, 1200, 1300, 1400, 1500, 1600, 1700, 1800, 1900, 2000, 2100, 2200, 2300, 2400 or 2500 μm), and preferably to approximately 100-150 μm. In some embodiments, the maximum depth ranges from 500 to 1000, 1000, 1100, 1200, 1300, 1400, 1500, 1600, 1700, 1800, 1900, 2000, 2100, 2200, 2300, 2400 or 2500 μm In one aspect, a needling device or needling adaptor described herein is suitable for disrupting skin to a depth of 100 μm. In one aspect, a needling device or needling adaptor described herein is suitable for disrupting skin to a depth of 150 μm.

In an example, the needles of the needling adaptor are arranged on a rectangular needle holder in two parallel or substantially parallel rows that extend across the rectangular needle holder. Substantially parallel as used herein means in the range of plus or minus 10%. In this example, there are 12 needles with 6 needles on a first row and 6 needles on a second row. Also, the needles of the first row are offset from the needles of the second row so that a needle on a first row is horizontally between and equidistant from two needles on a second row. In another example, three rows or more may be used. In an example, the needles are arranged in a rectangular arrangement so that the distance covered by the needles in one row is greater than the distance covered by the needles in one column. In another example, the needle holder may have a circular shape, and the needles may be arranged on the circular shaped holder.

Advantages of the needles and needle holder being arranged in a rectangular configuration include allowing a user or physician the ability to view the target region during the needling operation. The shape of the needle holder allows the user to follow a more precise needling pattern of a series of straight lines that do not overlap which also provides for a quicker and more efficient needling operation in contrast to a rounded arrangement of needles or a circular needle array.

By way of non-limiting example, and consistent with embodiments of the invention, the needling device, which in some embodiments may be a micropen used for micro-needling, includes a body, a power interface, charging contacts, a first dial for needle adjustment including a first measurement indicator, a second dial for needle adjustment including a second measurement indicator, and a light pipe for illuminating a light path in a direction parallel to the longitudinal axis of the micropen.

The micropen may include a rechargeable battery or other power unit in the upper section of the micropen body, a motor and drive mechanism between the first dial and the second dial, and a printed circuit board or control circuit above the first dial and at the area of power interface. In an example, a light pipe is included on the bottom surface of the micropen. This allows a user or operator to view the needling end of the device during a needling operation as light is projected from the light pipe through the adaptor.

The needling device may include one or more buttons for controlling a penetration depth or a speed of the needling operation or perturbations applied to a subject's skin, an on/off power button for the needling device, a power control of a light tube that allows light to exit from a distal end of the needling device to illuminate the perturbation region, and a trigger button, that is separate from the power button, for powering on the needling mechanism when pressed down by a user and powering off the needling mechanism when released by a user. In an example, needle penetration depth adjustment is controlled by a user rotating an adaptor cap which in turn rotates the micropen cap and a motor chassis.

Exemplary needling devices are described in International Patent Application PCT/US16/53972, published as WO/2017/054009, the content of which is herein incorporated by reference in its entirety, including the figures.

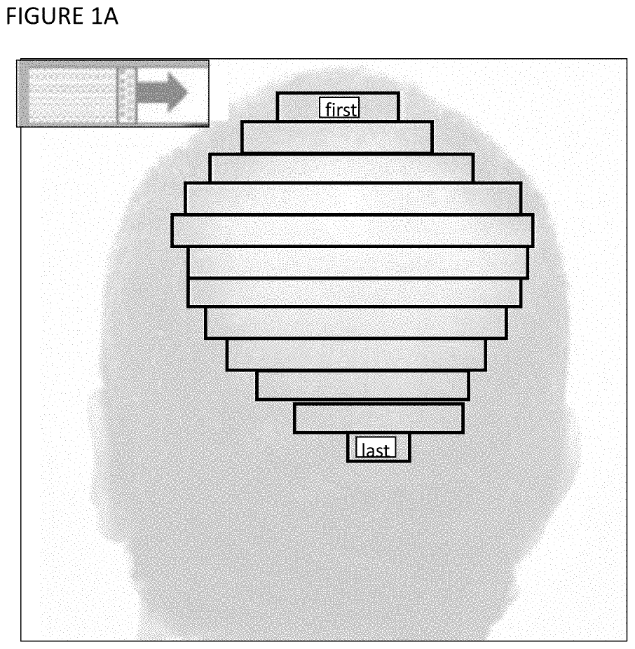

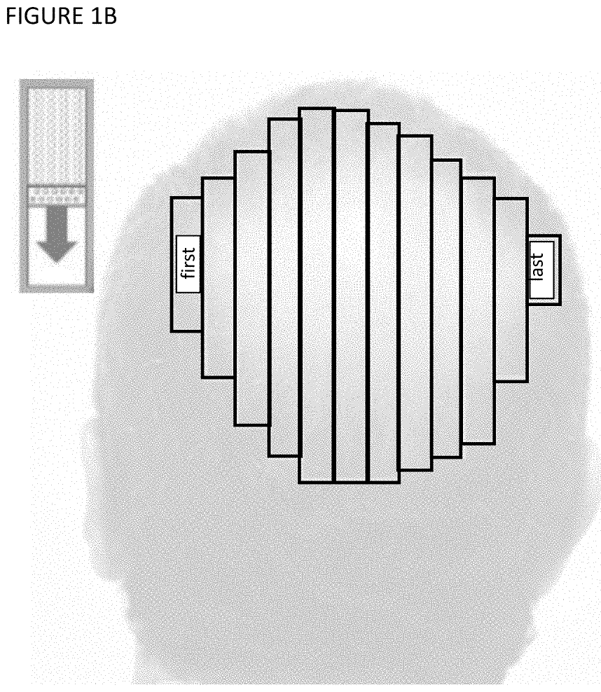

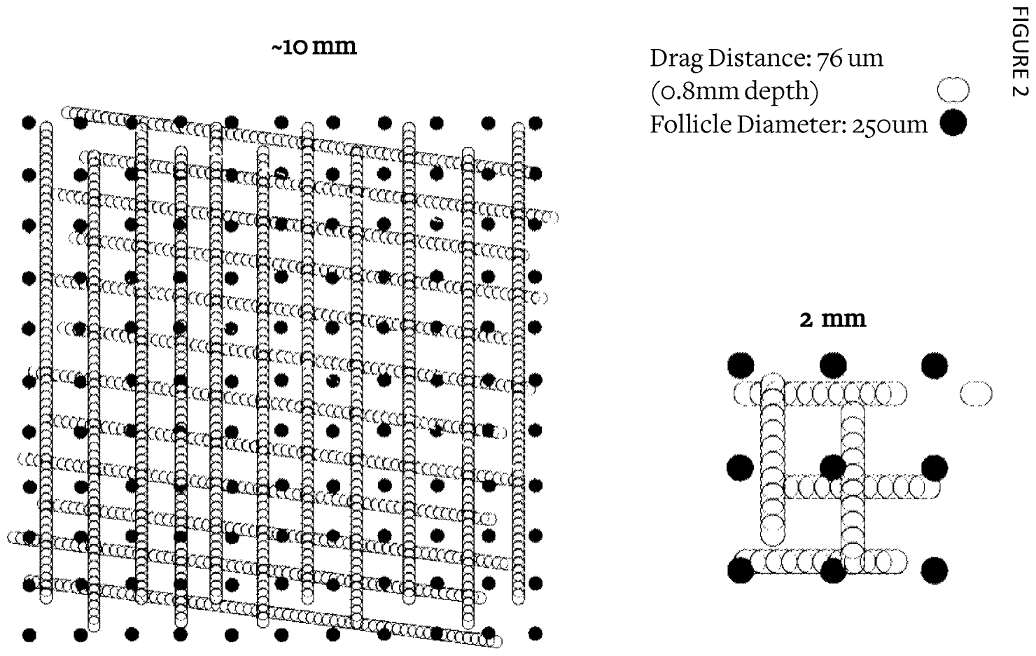

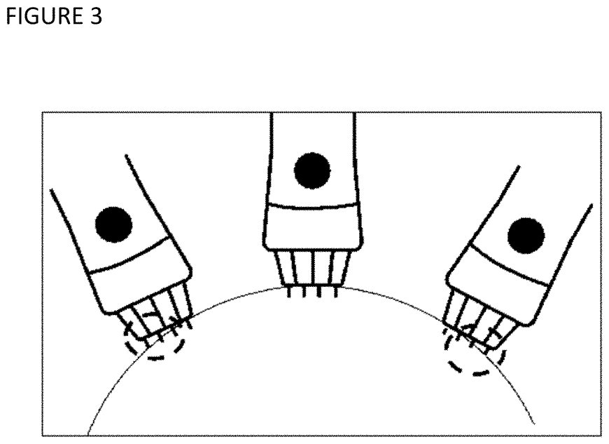

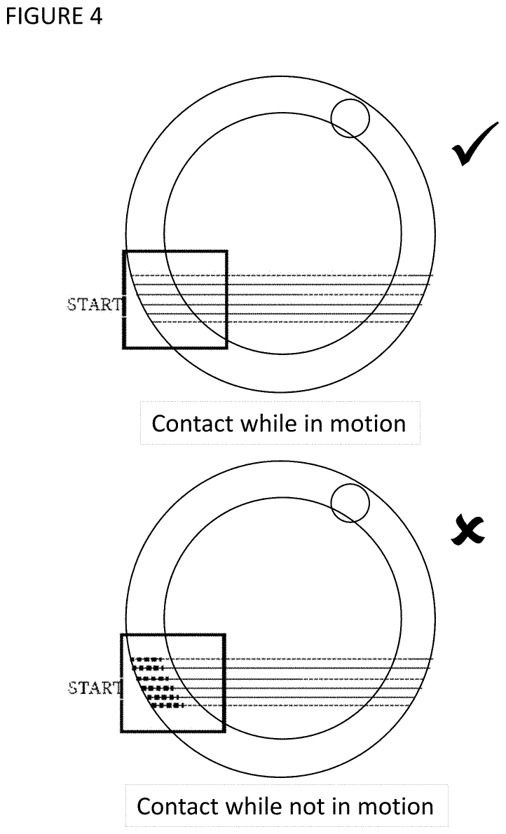

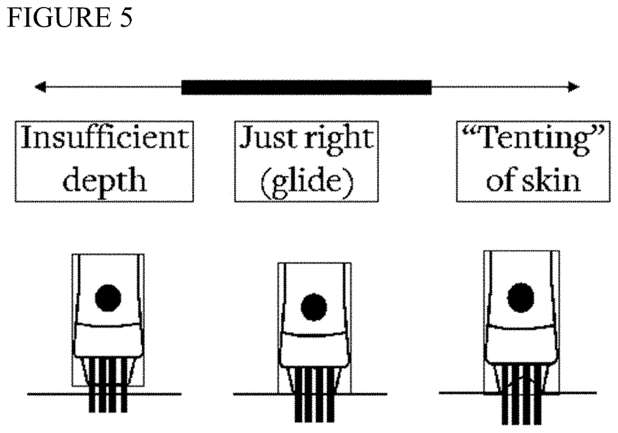

In some examples of using the device, the following parameters should be considered: speed, perpendicularity, contiguousness, initiation and complete of a treatment pass, pressure/contact and device orientation. Penetration depth may be uneven, if device is not perpendicular to the skin surface. Translation speed defines the density of the treatment. Faster or slower speeds impact the time to complete the procedure, the overall density, and wound healing (e.g., a slower speed may result in longer wound healing). In some embodiments of the disclosure, 2 perpendicular passes (e.g., horizontal and vertical) are performed (FIG. 1A-B, and FIG. 2). In some embodiments, the 2 perpendicular passes are performed at 1.5 to 2 cm/s, 2 to 2.5 cm/s, 2.5 to 3 cm/s, 3 to 3.5 cm/s, or 3.5 to 4 cm/s translation speed. In one embodiment, the 2 perpendicular passes are performed at about 2 cm/s translation speed. In one embodiment, the 2 perpendicular passes are performed at about 2 cm/s translation speed. In one embodiment, the needle strike has a consistent depth of 0.8 mm across the entire array. In order to achieve consistency, device is held perpendicular, thereby allowing even penetration depth of 0.8 mm (FIG. 3). In one embodiment, the drag distance is 76 um. When 2 perpendicular passes are performed, the second pass should align with the first pass, to ensure needling of the entire area to be treated and to avoid unneedled areas. To initiate a pass, the device should preferably contact the skin while in motion (“plane landing”, gliding start for each pass), as the active device is immediately engaged with the scalp at the point of contact, and the gliding motion avoids very high density at the stroke initiation (FIG. 4). The device should be smoothly retracted at the end of each pass. Consistent light pressure should be applied during each pass so as to allow the device to glide along the skin surface, and to avoid “tenting” of the skin (see FIG. 5). Consistent and light pressure ensures that the depth remains consistent and is not negatively impacted by “out of tolerance” pressure, and accordingly may have a positive impact on efficacy, tolerability and wound healing. Correct device orientation optimizes scalp coverage per pass and ensures optimal tolerability and wound healing. Incorrect orientation may result in less scalp coverage per pass, increased density, resulting in reduced tolerability and wound healing. In some examples, device alignment results in each pass consisting of six rows of two needles (6×2).

With respect to achieving integumental perturbation using micro-needling, the needle strike density is an important property of the treatment, as it is a significant determinant of key treatment outcomes, especially treatment effect size, procedure tolerability, and procedure safety. Several key treatment parameters interact to define needle strike density; these can be grouped into those determining (1) number of needle strikes, (2) treatment area, and (3) number of passes. In general, the goal is to achieve needle strikes that provide a uniform density of evenly spaced needle strikes with minimal distance between the strikes over a specified area of skin. An optimal strike density can be achieved using various combinations of parameters selected from: the number of needles in the needle array, the width of the needle array, the number of array oscillations per second, the device translation speed, and the treatment area. For example, in some embodiments, the needle strike density is 1600 needle strikes per cm2 or about 1600 needle strikes per cm2.

First, the number of (1) needle strikes per second can be calculated by multiplying: (1A) number of needles per needle array by (1B) the number of array oscillations per second. Second, the treatment area covered per second can be calculated by multiplying (2A) width of treatment (e.g., measured as the width of the needle array) by (2B) device translation speed. Overall, (1) the number of needle strikes, divided by (2) treatment area, multiplied by (3) the number of overlapping passes, results in the needle strike density. Several permutations of these treatment parameters exist in which equivalent treatment density is achieved.

In some embodiments of the methods described herein, one, two, three, four, or five passes are performed across the treatment area. In some embodiments, one pass is performed. In some embodiments, two passes are performed. In some embodiments, three passes are performed. In some embodiments of the methods described herein, the needle array has 4, 8, 12, 16, or 20 needles. In some embodiments of the methods described herein, the needle array has 5, 10, 15, 20, or 25 needles. In some embodiments of the methods described herein, the needle array has 6, 12, 18, 24, or 30 needles. In some embodiments of the methods described herein, the needle array has 7, 14, 21, 28, or 35 needles. In some embodiments of the methods described herein, the needle array has 8, 16, 24, 32, or 40 needles. In some embodiments, the needle array has 12 needles. In some embodiments, the needle array has 16 needles. In some embodiments, the needle array has 24 needles.

In some embodiments of the methods described herein the number of array oscillations per second is at least about 80 to 90, 90 to 100, 100 to 110, 110 to 115, 115 to 120, 120 to 125, 125 to 130, 130 to 140, or 140 to 150. In one embodiment, the number of array oscillations per second is 120. In some embodiments of the methods described herein, the width of treatment is at least about 0.4 to 0.5 cm, 0.5 to 0.6 cm, 0.6 to 0.7 cm, 0.7 to 0.8 cm, 0.8 to 0.9 cm, 0.9 to 1.0 cm, 1.0 to 1.1 cm, 1.1 to 1.2 cm, or 1.2 to 1.3 cm. In one embodiment, the width of treatment is at least about 0.9 cm. In one embodiment, the width of treatment is 0.9 cm.

In some embodiments of the methods described herein, the device translation speed is 1.5 to 2 cm/s, 2 to 2.5 cm/s, 2.5 to 3 cm/s, 3 to 3.5 cm/s, or 3.5 to 4 cm/s. In some embodiments of the methods described herein, the device translation speed is at least about 2 cm/s. In some embodiments of the methods described herein, the device translation speed is 2 cm/s. In some embodiments of the methods described herein, the strike density is 900 to 1000, 1000 to 1100, 1100 to 1200, 1200 to 1300, 1300 to 1400, 1400 to 1500, 1500 to 1600, 1600 to 1700, 1700 to 1800, 1800 to 1900, or 1900 to 2000 needle strikes per cm\hat{ }2. In some embodiments of the methods described herein, the strike density is 1550 to 1575, 1575 to 1600, 1600 to 1625, 1625 to 1650, or 1650 to 1700 needle strikes per cm\hat{ }2. In some embodiments of the methods described herein, the strike density is 1600 needle strikes per cm\hat{ }2.

For example, a treatment density of 1600 needle strikes per cm\hat{ }2 can be achieved by different combinations of the described parameters. In some embodiments of the methods described herein, the needle array has about 12 needles, and about 2 treatment passes are performed, wherein the number of array oscillations per second is set at about 120, the treatment width is about 0.9 cm, and the translation speed is about 2.0 cm/s. In some embodiments of the methods described herein, the needle array has 12 needles, and 2 treatment passes are performed, wherein the number of array oscillations per second is set at 120, the treatment width is 0.9 cm, and the translation speed is 2.0 cm/s.

In some embodiments of the methods described herein, the needle array has about 24 needles, and about 1 treatment pass is performed, wherein the number of array oscillations per second is set at about 120, the treatment width is about 0.9 cm, and the translation speed is about 2.0 cm/s. In some embodiments of the methods described herein, the needle array has 24 needles, and 1 treatment pass is performed, wherein the number of array oscillations per second is set at 120, the treatment width is 0.9 cm, and the translation speed is 2.0 cm/s.

In some embodiments of the methods described herein, the needle array has about 12 needles, and about 1 treatment pass is performed, wherein the number of array oscillations per second is set at about 120, the treatment width is about 0.9 cm, and the translation speed is about 1 cm/s. In some embodiments of the methods described herein, the needle array has 12 needles, and 1 treatment pass is performed, wherein the number of array oscillations per second is set at 120, the treatment width is 0.9 cm, and the translation speed is 1 cm/s.

In some embodiments of the methods described herein, the needle array has about 16 needles, and about 3 treatment passes are performed, wherein the number of array oscillations per second is set at about 90, the treatment width is about 1.8 cm, and the translation speed is about 1.5 cm/s. In some embodiments of the methods described herein, the needle array has 16 needles, and 3 treatment passes are performed, wherein the number of array oscillations per second is set at 90, the treatment width is 1.8 cm, and the translation speed is 1.5 cm/s.

### (II) Methods of Usings the Needling Device

In an example, methods of using a needling device include providing a needling device, having a sheath assembly with a needle array and a main unit including a motor for driving the needle array, opening the sheath assembly and placing the main unit within the sheath assembly so that the main unit is fully encapsulated and protected from the outside environment; and powering on the needling device. The method may further include removing the sheath assembly and replacing the sheath assembly with another sheath assembly having a different needle array.

For example, a needle array having a rectangular configuration may be provided on a first sheath adaptor that is used by a physician on a patient for hair growth applications. After use, the first adaptor may be replaced by a second sheath adaptor having a different needle array configuration, for example, a circular needle array configuration. The needling device may be used on different parts of one patient's skin without a need to clean the needling device because it is fully encapsulated within the adaptor sheath. Also, the needling device may be used on different patients. For example, a physician may use the first needling adaptor with the device on a first patient and then remove and replace the first adaptor with a second needling adaptor for use on a second patient.

In a specific embodiment, the needling device is used in a method of treatment described in Section 6.3.1.

### 6.3 METHODS OF INTEGUMENTAL PERTURBATION AND ADMINISTRATION OF VALPROIC ACID TOGETHER

In some embodiments, the methods described herein comprises administration of valproic acid or a pharmaceutically acceptable salt thereof in combination with integumental perturbation.

In some embodiments, the invention provides a method for enhancing hair growth in a patient with scarring alopecia comprising controlled integumental perturbation using a fractional ablative laser, followed by twice daily topical administration of a pharmaceutical composition (e.g., a pharmaceutical composition comprising valproic acid or a pharmaceutically acceptable salt thereof) for 14 days. In certain embodiments, the administration of a pharmaceutical composition (e.g., a pharmaceutical composition comprising valproic acid or a pharmaceutically acceptable salt thereof) is begun on the same day as the laser treatment.

In some embodiments, the pharmaceutical composition is administered before and after integumental perturbation. In some embodiments, the pharmaceutical composition is administered once reepithelialization is completed, or 1, 2, 3, 4, 5, 6, 7, 8, 9, 10, 11, 12, 13, 14, 15, or 16 weeks after integumental perturbation.

In some embodiments, the pharmaceutical composition comprising valproic acid or a pharmaceutically acceptable salt thereof is administered 1, 2, 3, or more weeks after integumental perturbation. In some embodiments, the pharmaceutical composition comprising valproic acid or a pharmaceutically acceptable salt thereof is administered 2, 4, 6, 8, 10, 12, 14, 16, 18, 20, 22, or 24 hours; or 1, 2, 3, 4, 5, 6, or 7 days after integumental perturbation. For example, in an embodiment, the pharmaceutical composition comprising valproic acid or a pharmaceutically acceptable salt thereof is administered 1 week after integumental perturbation.

In some embodiments, the pharmaceutical composition comprising valproic acid or a pharmaceutically acceptable salt thereof is administered for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. In some embodiments, the pharmaceutical composition comprising valproic acid or a pharmaceutically acceptable salt thereof is administered for 1, 2, 3, or 4 weeks; 1, 2, 3, 4, 5, or 6 months. In some embodiments, the pharmaceutical composition comprising valproic acid or a pharmaceutically acceptable salt thereof is administered until a desired biological outcome is achieved (e.g., a biological outcome as described in Section 6.4.1)

In some embodiments, a course of therapy comprises integumental perturbation of an area of the skin of a human subject where hair growth is desired; and administering a pharmaceutical composition comprising valproic acid or a pharmaceutically acceptable salt, isotopic variant, or solvate thereof, multiple times. For example, a course of therapy can include performing integumental perturbation and administering a pharmaceutical composition comprising valproic acid or a pharmaceutically acceptable salt 3, 4, 5, 6, 7, 8, 9, 10, 11, or 12 times. In some embodiments, the course of therapy comprises performing integumental perturbation and administering a pharmaceutical composition comprising valproic acid or a pharmaceutically acceptable salt 3, 6, or 12 times. In some embodiments, the course of therapy comprises performing integumental perturbation monthly, biweekly, or weekly. In some embodiments, the course of therapy occurs over 1, 2, or 3 months. In some embodiments, the course of therapy comprises a baseline integumental perturbation on day 1. In some embodiments, the course of therapy comprises integumental perturbation every 4 weeks. For example, in some embodiments, the course of therapy comprises integumental perturbation on days 1, 29, and 57. In some embodiments, the course of therapy comprises integumental perturbation every 2 weeks. For example, in some embodiments, the course of therapy comprises integumental perturbation on days 1, 15, 29, 43, 57, and 71. In some embodiments, the course of therapy comprises integumental perturbation every week. For example, in some embodiments, the course of therapy comprises integumental perturbation on days 1, 8, 15, 22, 29, 36, 43, 50, 57, 64, 71, and 78.

In some embodiments, the pharmaceutical composition comprising valproic acid or a pharmaceutically acceptable salt thereof is administered until 24 hours before a second course of treatment. In some embodiments, the pharmaceutical composition comprising valproic acid or a pharmaceutically acceptable salt thereof is administered until 24 hours before a second integumental perturbation. In some embodiments, valproic acid or a pharmaceutically acceptable salt thereof is administered in combination with an additional agent (e.g., an additional agent as described in Section 6.5, e.g., minoxidil). In some embodiments, a second course of treatment follows at least 24 hours after a first course of treatment.

### 6.3.1 Methods of Using the Needling Device and Applicator Device Together

A method of using needling devices and drug applicators together for stimulating hair growth, may include providing a needling device having a sheath assembly including a needle array; and a main unit comprising a motor for driving the needle array; providing a drug applicator for drug delivery and massaging, including a housing; a drug delivery cartridge carried by the housing; and a massage head which is mounted on the housing for massaging a subject's skin; using the needling device to perform targeted cutaneous perturbation for disrupting a layer of a human scalp; and after using the needling device, using the drug applicator for applying a drug to the disrupted layer of the human scalp.

The method may further include disrupting the layer of the human scalp that is the basal or suprabasal epidermal layer, with the drug that is being applied being valproic acid or a pharmaceutical acceptable salt, as described in Section 6.1.2.

In some embodiments, the needling device and applicator device are used in a method of treatment described in Section 6.4.

### 6.4 METHODS OF PROPHYLACTIC AND THERAPEUTIC USE

Provided herein is a method of treatment, comprising (a) integumental perturbation of an area of skin of a subject in need thereof, wherein the integumental perturbation is performed by a needling device and/or adaptor described herein; and (b) after a first period of time, administering to the subject a first pharmaceutically effective dose of valproic acid or a pharmaceutical acceptable salt as described in Section 6.1.2 or formulation thereof, wherein the method of treatment achieves one or more biological outcome described in Section 6.4.1. In a specific embodiment, the method further comprises: (c) after a second period of time, administering to the subject a second pharmaceutically effective amount of the agent or formulation thereof. In a specific embodiment, step (c) of the method is repeated 1, 2, 3, 4, 5, 6, 7, 8, 9, 10 or more times or until an outcome described in Section 6.4.1 is achieved. In a specific embodiment, the integumental perturbation is as described in Section 6.2.

In a specific embodiment, provided herein is a method of treatment, comprising administering a first pharmaceutically effective dose of valproic acid or a pharmaceutical acceptable salt as described in Section 6.1.2 or formulation thereof to an area of skin of a subject in need thereof, wherein the area of skin is integumentally perturbed. In a specific embodiment, valproic acid or a pharmaceutical acceptable salt, or formulation thereof is administered to the subject after a first period of time, wherein the first period of time is the time since the skin was integumentally perturbed. In a specific embodiment, the method further comprises: (c) after a second period of time, administering to the subject a second pharmaceutically effective amount of valproic acid or a pharmaceutical acceptable salt or formulation thereof. In a specific embodiment, step (c) of the method is repeated 1, 2, 3, 4, 5, 6, 7, 8, 9, 10 or more times or until an outcome described in Section 6.4.1 is achieved. In a specific embodiment, the integumental perturbation is as described in Section 6.2.

In a specific embodiment, the area of skin is an area of a subject in which hair growth is desired, for example, the scalp, the face (e.g., the eyebrow, eyelashes, upper lip, lower lip, chin, cheeks, beard area, or mustache area), or another part of the body, such as, e.g., the chest, abdomen, arms, armpits (site of auxillary hair), legs, or genitals. In a specific embodiment, the area of skin is the head. In a specific embodiment, the area of skin is the scalp. In some embodiments, the area of skin is a balding scalp. In a specific embodiment, the area of skin is not on the face. In a specific embodiment, the area of skin is not on an area of the skin that is normally covered with only, or mostly, vellus hair. In a specific embodiment, hair restoration to a wounded or scarred part of the skin is desired and/or scar revision is desired. Thus, in a specific embodiment, the area of skin is a wounded or scarred part of the skin. In a specific embodiment, the scar is caused by surgery, such as a face lift, skin graft, or hair transplant.

In a specific embodiment, the area of skin is an area of skin of any desired size, for example, between 0-3 mm in width (e.g., 1 mm, 2 mm, 3 mm, or greater), 0-2 cm in width (e.g., 1 cm, 1.5 cm, and 2.0 cm), or greater (for example, up to 10%, 30%, 50%, 70%, 90%, or 100% of a subject's skin). Optionally, the area of skin is interfollicular.

In a specific embodiment, the first period of time is less than 1 minute, 2 minutes, 3 minutes, 4 minutes, 5 minutes, 10 minutes, 15 minutes, 20 minutes, 25 minutes, 30 minutes, 45 minutes, 60 minutes, 3 hours, 6 hours, 12 hours, 24 hours, 2 days, 3 days, 4 days, 5 days, 6 days, 7 days, 8 days, 9 days, 10 days, 11 days, 12 days, 13 days, 14 days, 3 weeks, or 4 weeks. In a specific embodiment, the first period of time between is at least 1 minute, 2 minutes, 3 minutes, 4 minutes, 5 minutes, 10 minutes, 15 minutes, 20 minutes, 25 minutes, 30 minutes, 45 minutes, 60 minutes, 3 hours, 6 hours, 12 hours, 24 hours, 2 days, 3 days, 4 days, 5 days, 6 days, 7 days, 8 days, 9 days, 10 days, 11 days, 12 days, 13 days, 14 days, 3 weeks, or 4 weeks. In a specific embodiment, the first period of time between is at most 1 minute, 2 minutes, 3 minutes, 4 minutes, 5 minutes, 10 minutes, 15 minutes, 20 minutes, 25 minutes, 30 minutes, 45 minutes, 60 minutes, 3 hours, 6 hours, 12 hours, 24 hours, 2 days, 3 days, 4 days, 5 days, 6 days, 7 days, 8 days, 9 days, 10 days, 11 days, 12 days, 13 days, 14 days, 3 weeks, or 4 weeks.

In a specific embodiment, valproic acid or a pharmaceutically acceptable salt thereof is administered to the subject via an applicator device described herein.

In a specific embodiment, valproic acid or a pharmaceutically acceptable salt thereof is administered to the area of skin on the subject on which the needling device was used.

In a specific embodiment, the second period of time is less than 1 minute, 2 minutes, 3 minutes, 4 minutes, 5 minutes, 10 minutes, 15 minutes, 20 minutes, 25 minutes, 30 minutes, 45 minutes, 60 minutes, 3 hours, 6 hours, 12 hours, 24 hours, 2 days, 3 days, 4 days, 5 days, 6 days, 7 days, 8 days, 9 days, 10 days, 11 days, 12 days, 13 days, 14 days, 3 weeks, or 4 weeks. In a specific embodiment, the second period of time at least 1 minute, 2 minutes, 3 minutes, 4 minutes, 5 minutes, 10 minutes, 15 minutes, 20 minutes, 25 minutes, 30 minutes, 45 minutes, 60 minutes, 3 hours, 6 hours, 12 hours, 24 hours, 2 days, 3 days, 4 days, 5 days, 6 days, 7 days, 8 days, 9 days, 10 days, 11 days, 12 days, 13 days, 14 days, 3 weeks, or 4 weeks. In a specific embodiment, the second period of time is at most 1 minute, 2 minutes, 3 minutes, 4 minutes, 5 minutes, 10 minutes, 15 minutes, 20 minutes, 25 minutes, 30 minutes, 45 minutes, 60 minutes, 3 hours, 6 hours, 12 hours, 24 hours, 2 days, 3 days, 4 days, 5 days, 6 days, 7 days, 8 days, 9 days, 10 days, 11 days, 12 days, 13 days, 14 days, 3 weeks, or 4 weeks.

In a specific embodiment, the first pharmaceutically effective dose of valproic acid or a pharmaceutically acceptable salt thereof is a dose described in Section 6.1.1. In a specific embodiment, the second pharmaceutically effective dose of valproic acid or a pharmaceutically acceptable salt thereof is a dose described in Section 6.1.1.

In some embodiments, the integumental perturbation induces a wound in the skin. In a specific embodiment, the integumentally perturbed skin is wounded. In some such embodiments, the wounded skin is healed by primary intention. In other embodiments, the wounded skin is healed by secondary intention. In yet other embodiments, the wounded skin is healed by tertiary intention. In certain embodiments, the wounded skin is healed more slowly than usually indicated for that kind of wound. This may enhance scarless wound healing and/or prolong the period during which hair growth in the wounded area of skin is promoted. In a specific embodiment, the method further comprises administering a post-perturbation wound healing compound.

In a specific embodiment, the subject is a subject described in Section 6.4.2.

### 6.4.1 BIOLOGICAL OUTCOMES

In a specific embodiment, the methods described herein are suitable for achieving one or more of the biological outcomes described herein.

In a specific embodiment, the methods described herein are suitable to achieve one or more of the nonlimiting examples of biological outcomes described in Section 6.4.1 in a subject on which the methods are used: hair growth applications. Nonlimiting examples of hair growth applications include increasing the amount of hair, increasing hair thickness, increasing hair longevity, inducing hair follicle neogenesis, treating baldness, treating alopecia, promoting hair follicle development and/or activation on an area of the skin of the subject.

In a specific embodiment, the methods described herein are suitable to achieve one or more of the following nonlimiting examples of biological outcomes in a subject on which the methods were used: to promote generation of new hair follicles (“follicle neogenesis”); to promote formation of neogenic-like (NL) follicular structures; to promote activation (possibly by reorganization) of existing hair follicles; to promote formation of pre-existing-like (PEL) or pre-existing-like, attached (PELA) follicular structures; to promote development of hair follicles, for example, to promote the growth of non-vellus hair (in preference to vellus hair); to promote the growth of terminal hair (in preference to vellus hair); to promote the branching of pre-existing hair follicles (seen as an increased number of hair shafts per pore); to increase the width of hair follicles (thereby promoting growth of an increased shaft width); to delay or prevent follicle senescence;

In a specific embodiment, the methods described herein are suitable to achieve one or more of the following nonlimiting examples of biological outcomes in a subject on which the methods were used: to promote the growth of hair; to promote growth of vellus hair; to promote the transition of vellus hair to non-vellus hair; to promote the transition of vellus hair to terminal hair; to increase the amount of hair follicles in anagen, to prolong anagen, to shorten telogen, to promote growth of non-vellus hair; to increase the amount of hair follicles in anagen, to prolong anagen, to shorten telogen, to promote growth of terminal hair; to increase the amount of hair; to increase the thickness of hair; and/or to reduce or prevent hair loss.

In one embodiment, the biological outcome is growth of hair on the area of skin of a subject. In some embodiments, the biological outcome is an increase in the amount or thickness of hair on a treated area of skin of a subject. In some embodiments, the biological outcome is an increase in the amount of vellus hair on a treated area of skin of a subject. In some embodiments, the biological outcome is an increase in the amount of non-vellus hair on a treated area of skin of a subject. In some embodiments, the biological outcome is the maintenance of non-vellus hair growth, i.e. helps prevent miniaturization of terminal hairs. In some embodiments, the biological outcome is an increase in the ratio of non-vellus -to-vellus hair on a treated area of skin of a subject. In some embodiments, the biological outcome is an increase in the amount of terminal hair on a treated area of skin of a subject. In some embodiments, the biological outcome is the maintenance of terminal hair growth, i.e. helps prevent miniaturization of terminal hairs. In some embodiments, the biological outcome is an increase in the ratio of terminal-to-vellus hair on a treated area of skin of a subject. In some embodiments, the biological outcome is an increase in the amount of anagen hair or increases anagen growth on a treated area of skin of a subject. In some embodiments, the biological outcome is an increase in the ratio of anagen-to-telogen hair on a treated area of skin of a subject. In some embodiments, the biological outcome is hair follicle neogenesis in a treated area of skin of a subject. In some embodiments, the biological outcome is an increased number of hair follicles in a treated area of skin of a subject. In some embodiments, the biological outcome is formation of new hair follicles with non-vellus-sized hair shafts (i.e., hair shafts with diameters equal to or greater than 30 microns in diameter) in a treated area of skin of a subject. In some embodiments, the biological outcome is an increased number of stimulated and activated hair follicles, such as pre-existing hair follicles, in a treated area of skin of a subject. In some embodiments, the biological outcome is an increased number of pre-existing hair follicles with non-vellus-sized hair shafts in a treated area of skin of a subject. In some embodiments, the biological outcome is the presence and/or increased numbers of neogenic-like (NL) follicular structures, pre-existing-like (PEL), and -existing-like, attached (PELA) follicular structures.

Success of methods to achieve one or more of the biological outcomes described herein and/or success of a method of the invention can be measured by, for example: (i) improved overall cosmetic outcome (e.g., using the Visual Analogue Scale (VAS)); (ii) patient assessment of his/her hair growth (e.g., based on questionnaire); (iii) investigator assessment of hair growth in a patient (e.g., based on a rating scale); (iv) patient assessment of his/her hair growth in photographs; (v) investigator assessments of hair growth in patient photographs; (vi) increased hair count (e.g., by measuring new hair growth as an increased number of fibers in an affected area of the skin); (vii) increased hair density; (viii) increased thickness of hair or hair shaft (e.g., based on diameter); (ix) increased hair weight; (x) hair cuttings; (xi) longer hair; (xii) increase in the amount of terminal hair (by, e.g., measuring new hair growth as an increased number of fibers in an affected area of the skin, or increased thickness (e.g., diameter) or length of hair fibers); (xiii) increase in the amount of vellus hair (by, e.g., measuring new hair growth as an increased number of fibers in an affected area of the skin) (e.g., as measured photographically); (xiv) increase in the amount of non-vellus hair, e.g., intermediate or terminal hair; (xv) an increase in the ratio of terminal-to-vellus hair; or an increase in the ratio of non-vellus-to-vellus hair; (xvi) increased number of hair germs; (xvii) increased number of hair follicles (e.g., as evaluated by a skin biopsy); (xviii) increased number of hair follicles at a more mature stage of development; (xix) increased numbers of follicular units with 3 or more hair follicles; (xx) increased hair follicle branching; (xxi) formation of new hair follicles (“hair follicle neogenesis”); (xxii) formation of new hair follicles with vellus-sized hair shafts (i.e., hair shafts with diameters less than 30 microns in diameter); (xxiii) formation of new hair follicles with non-vellus-sized hair shafts (i.e., hair shafts with diameters 30 microns or greater in diameter); (xxiv) hair follicle regeneration; (xxv) increased activation of existing hair follicles; (xxvi) increased number of hair follicles; (xxvii) increased number of activated hair follicles; (xxviii) increased number of activated pre-existing hair follicles; (xxix) presence or increased numbers of neogenic-like (NL) hair follicles (based on, e.g., examination of a biopsy or by confocal microscope, by assessing number of hair follicles, and/or by assessing morphology of hair follicles compared to baseline or a negative control); (xxx) presence or increased numbers of pre-existing hair follicles (based on, e.g., examination of a biopsy or by confocal microscope, by assessing number of hair follicles, and/or by assessing morphology of hair follicles compared to baseline or a negative control); (xxxi) presence or increased numbers of primitive structures of interest (SOIs), such as neogenic-like (NL), pre-existing-like (PEL), and/or pre-existing-like, attached (PELA) follicular structures (based on, e.g., examination of a biopsy or by confocal microscope, by assessing number of hair follicles, and/or by assessing morphology of hair follicles compared to baseline or a negative control, as described for example in Section 5.8.4 infra); (xxxii) increased number of pre-existing hair follicles with vellus-sized hair shafts in a treated area of skin of a subject; (xxxiii) increased number of neogenic-like hair follicles with vellus-sized hair shafts in a treated area of skin of a subject; (xxxiv) increase in the amount of anagen hair; (xxxv) increase in the amount of telogen hair; (xxxvi) increased proportion of hair follicles in anagen or decreased proportion of hair follicles in telogen (i.e., an increase in the ratio of anagen-to-telogen hair) (based on, e.g., examination of a biopsy or phototrichogram); (xxxvii) increased proliferation of dermal papilla (based on, e.g., examination of a biopsy); and/or (xxxviii) increased recruitment or proliferation of stem cells to the follicle (based on, e.g., examination of a biopsy).

In certain embodiments, the methods described herein are suitable for achieving uniform integumental perturbations. For example, in certain embodiments, the methods described herein are suitable for achieving uniformity in the perforations of the integumental perturbation. In a specific embodiment, uniformity is measured by the variance in perforations per square centimeter. In a specific embodiment, the uniformity is at least +/−1%, at least +/−3%, at least +/−5%, at least +/−10%, at least +/−15%, at least +/−20%, at least +/−30%, at least +/−50%. In a specific embodiment, the uniformity is at most +/−1%, at most +/−3%, at most +/−5%, at most +/−10%, at most +/−15%, at most +/−20%, at most +/−30%, at most +/−50%, at most +/−60%, at most +/−70%, at most +/−80%, or at most 90%.

In certain embodiments, success of treatment is assessed by examination of hair follicles in a treated area of the subject's skin. In certain embodiments, hair follicles are examined histologically, or by determination of the presence or absence of certain markers of hair follicle development or morphology. The area of skin for examination may be obtained by biopsy, such as a punch biopsy; alternatively or in addition, in a less invasive method, the skin may be analyzed directly by, e.g., confocal microscopy or other technique that permits imaging beneath the surface of the skin.

In some embodiments, an increase in stimulated, activated, and reorganized follicular structures or new follicle formation may be assessed post biopsy using immunofluorescence techniques for the detection of markers of associated with follicle neogenesis. For example, levels of known stimulators of neofollicle formation, e.g., WNT pathway proteins, e.g., beta-catenin, may be analyzed to assess initiation or progression of hair follicle activation or neogenesis. Accordingly, in some embodiments, at 1, 2 or 3 weeks after use of the methods described, analysis of material from a biopsy taken from the area of interest, e.g., the scalp, shows increased WNT pathway activation (e.g., increased levels of beta-catenin) compared to material from a biopsy taken immediately before the use of the methods described.

Entry into the anagen phase is also regulated by bone morphogenetic protein (BMP), Sonic hedgehog (Shh), fibroblast growth factor (FGF), and transforming growth factor (TGF)-β. Accordingly, in some embodiments, levels of signaling proteins within these pathways may be used to analyze an increase in stimulated, activated, and reorganized follicular structures or new follicle formation. In some embodiments, at 1, 2 or 3 weeks after use of the methods described, analysis of material from a biopsy taken from the area of interest, e.g., the scalp, shows increased SSH pathway activation (e.g., increased SSH) compared to material from a biopsy taken immediately before the use of the methods described. In some embodiments, at 1, 2 or 3 weeks after use of the methods described, analysis of material from a biopsy taken from the area of interest, e.g., the scalp, shows increased FGF pathway activation (e.g., Akt phosphorylation) compared to material from a biopsy taken immediately before the use of the methods described. In some embodiments, at 1, 2 or 3 weeks after use of the methods described, analysis of material from a biopsy taken from the area of interest, e.g., the scalp, shows increased TGF-β pathway activation (e.g., Smad phosphorylation) compared to material from a biopsy taken immediately before the use of the methods described.

At the beginning of the anagen phase, secondary hair germ cells rapidly proliferate and produce transit amplifying cells in the germinal matrix of the epidermis. Next, stem cells in the bulge region proliferate and generate outer root sheath cells. Accordingly, proliferation markers may be used to detect an increase in stimulated, activated, and reorganized follicular structures or new follicle formation. Non-limiting examples of a proliferation marker, which may be detected using immune staining techniques include Ki67 and PCNA. In some embodiments, at 1, 2 or 3 weeks after use of the methods described, analysis of material from a

Attorney Docket No. 12718-064-999 biopsy taken from the area of interest, e.g., the scalp, shows increased proliferation markers compared to material from a biopsy taken immediately before the use of the methods described.

Alternatively, markers for hair follicle stem cells may be used, e.g., CD34+, keratin 15, and Sox9 (Woo et al., SnapShot: Hair Follicle Stem Cells Cell. 2011 Jul 22; 146(2): 334-334.e2). Accordingly, in some embodiments, at 1, 2 or 3 weeks after use of the methods described, analysis of material from a biopsy taken from the area of interest, e.g., the scalp, shows an increase in one or more hair follicle stem cell markers compared to material from a biopsy taken immediately before the use of the methods described.

Several other markers for hair growth (e.g., stimulated, activated, and reorganized follicular structures or new follicle formation) are known in the art, e.g., proteins expressed in dermal papilla cells (DP) (Yang and Cotsarellis, Review of hair follicle dermal cells; J Dermatol Sci. 2010 Jan; 57(1): 2). For example, the activity of alkaline phosphatase (AP) has been used as a marker to detect the presence of DP and is regarded as an indicator for hair inductivity; AP activity in DP reach its maximal level in early anagen, and decrease after mid-anagen growing phase. In human hair follicles, versican is reported specifically expressed in DP during anagen, and is considered a suitable marker for the anagen stage. CD133 is a hematopoietic stem cell marker that is strongly expressed in DP of stage 3-4 developing hair follicles and may also be used alone or in combination to detect anagen stage. Accordingly, in some embodiments, at 1, 2 or 3 weeks after use of the methods described, analysis of material from a biopsy taken from the area of interest, e.g., the scalp, shows an increase in one or more DP markers described herein (e.g., AP, versican, and/or CD133) or known in the art compared to material from a biopsy taken immediately before the use of the methods described.

In any of these embodiments, an increase in levels of a biomarker of hair growth described above or otherwise known in the art is at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80-85%, 85-90%, 90-95%, or at least 95-100% greater compared to immediately before the use of the methods described. In any of these embodiments, an increase in levels of a biomarker of hair growth described above or otherwise known in the art is at least 5%, 10%, 15%, 20%, 25%, 30%, 35%, 40%, 45%, 50%, 55%, 60%, 65%, 70%, 75%, 80%, 85%, 90%, 95%, or at least 100% greater compared to immediately before the use of the methods described.

In one embodiment, an increase in levels of alkaline phosphatase is at least 1-5%, 5-10%, 10-15%, 15-20%, 20-25%, 25-30%, 30-35%, 35-40%, 40-45%, 45-50%, 50-55%, 55-60%, 60-65%, 65-70%, 70-75%, 75-80%, 80% -85%, 85-90%, 90-95%, or at least 95-100% greater compared to immediately before the use of the methods described. In one embodiment, an increase in levels of alkaline phosphatase is at least 5%, 10%, 15%, 20%, 25%, 30%, 35%, 40%, 45%, 50%, 55%, 60%, 65%, 70%, 75%, 80%, 85%, 90%, 95%, or at least 100% greater compared to immediately before the use of the methods described.

### 6.4.2 Subject Populations

The methods described herein are suitable for use on subject described herein. Thus, described herein are candidate subjects for treatment with a method described herein. In a specific embodiment, the subject is any subject suffering from hair loss, hair thinning, balding, or who has or has had a disease or condition associated therewith, or who wishes to enhance the growth or thickness of hair or prevent hair loss.

The subject may be any subject, preferably a human subject, including male, female, intermediate/ambiguous (e.g., XO), and transsexual subjects. In certain embodiments, the subject is a human adolescent. In certain embodiments, the subject is undergoing puberty. In certain embodiments, the subject is a middle-aged adult. In certain embodiments, the subject is a premenopausal adult. In certain embodiments, the subject is undergoing menopause. In certain embodiments, the subject is elderly. In certain embodiments, the subject is a human of 1 year old or less, 2 years old or less, 2 years old, 5 years old, 5 to 10 years old, 10 to 15 years old, e.g., 12 years old, 15 to 20 years old, 20 to 25 years old, 25 to 30 years old, 30 years old or older, 30 to 35 years old, 35 years old or older, 35 to 40 years old, 40 years old or older, 40 to 45 years old, 45 to 50 years old, 50 years old or older, 50 to 55 years old, 55 to 60 years old, 60 years old or older, 60 to 65 years old, e.g., 65 years old, 65 to 70 years old, 70 to 75 years old, 75 to 80 years old, 80 to 85 years old, 85 to 90 years old, 90 to 95 years old or 95 years old or older. In some embodiments, the subject is a male 20 to 50 years old. In some embodiments, the subject is a male 20 to 60 years old. In some embodiments, the subject is a male 30 to 60 years old. In some embodiments, the subject is a male 40 to 60 years old. In some embodiments, the subject is a male or female 12 to 40 years old. In some embodiments, the subject is not a female subject. In some embodiments, the subject is not pregnant or expecting to become pregnant. In some embodiments, the subject is not a pregnant female in the first trimester of pregnancy. In some embodiments, the subject is not breastfeeding.

In one embodiment, the treatment is delivered to an area in which hair growth is desired, for example, the scalp, the face (e.g., the eyebrow, eyelashes, upper lip, lower lip, chin, cheeks, beard area, or mustache area), or another part of the body, such as, e.g., the chest, abdomen, arms, armpits (site of axillary hair), legs, or genitals. In some embodiments, treatment is delivered to the head. In some embodiments, treatment is delivered to the scalp. In some embodiments, treatment is delivered to a balding scalp. In one embodiment, treatment is not delivered to the face. In one embodiment, treatment is not delivered to an area of the skin that is normally covered with only, or mostly, vellus hair. In some embodiments, hair restoration to a wounded or scarred part of the skin is desired. In one embodiment, the scar is caused by surgery, such as a face lift, skin graft, or hair transplant.

The subject may have a disease or disorder of balding or hair loss (including hair thinning), such as forms of nonscarring (noncicatricial) alopecia, such as androgenetic alopecia (AGA), including male-patterned hair loss (MPHL) or female-patterned hair loss (FPHL) (e.g., thinning of the hair, i.e., diffuse hair loss in the frontal/parietal scalp), or any other form of hair loss caused by androgens, toxic alopecia, alopecia areata (including alopecia universalis), scarring (cicatricial) alopecia, pathologic alopecia (caused by, e.g., medication, trauma stress, autoimmune diseases, malnutrition, or endocrine dysfunction), trichotillomania, a form of hypotrichosis, such as congenital hypotrichosis, lichen planopilaris, or Central Centrifugal Cicatricial Alopecia (CCCA) or any other condition of hair loss or balding known in the art or described infra.

In some embodiments, the subject has hair loss caused by a genetic or hereditary disease or disorder, such as androgenetic alopecia.

In some embodiments, the subject has hair loss caused by anagen effluvium, such as occurs during chemotherapy (with, e.g., 5-fluorouracil, methotrexate, cyclophosphamide, vincristine). In addition to chemotherapy drugs, Anagen effluvium can be caused by other toxins, radiation exposure (including radiation overdose), endocrine diseases, trauma, pressure, and certain diseases, such as alopecia areata (an autoimmune disease that attacks anagen follicles.)

In some embodiments, the subject has hair loss caused by telogen effluvium. Telogen effluvium is caused frequently by drugs like lithium and other drugs like valproic acid and carbamazepine. In addition to psychiatric drugs, telogen effluvium can be induced by childbirth, traction, febrile illnesses, surgery, stress, or poor nutrition. (See, Mercke et al., 2000, Ann. Clin. Psych. 12:35-42).

In some embodiments, the subject has hair loss caused by or associated with medication, such as chemotherapy (e.g., anti-cancer therapy or cytotoxic drugs), thallium compounds, vitamins (e.g., vitamin A), retinoids, anti-viral therapy, or psychological therapy, radiation (such as the banding pattern of scalp hair loss that may be caused by radiation overdose), trauma, endocrine dysfunction, surgery, physical trauma, x-ray atrophy, burning or other injury or wound, stress, aging, an autoimmune disease or disorder, malnutrition, an infection (such as, e.g., a fungal, viral, or bacterial infection, including chronic deep bacterial or fungal infections), dermatitis, psoriasis, eczema, pregnancy, allergy, a severe illness (e.g., scarlet fever), myxedema, hypopituitarism, early syphilis, discoid lupus erythematosus, cutaneous lupus erythematosus, lichen planus, deep factitial ulcer, granuloma (e.g., sarcoidosis, syphilitic gummas, TB), inflamed tinea capitis (kerion, favus), a slow-growing tumor of the scalp or other skin tumor, or any other disease or disorder associated with or that causes balding or hair loss known in the art or described infra.

In some embodiments, the subject has hair thinning, or “shock loss,” or a bald patch caused by prior use as a source of tissue or follicles for hair transplantation or follicular unit transplantation.

In some embodiments, a candidate subject is a subject who wishes to enhance hair growth, for example, to have more hair, faster-growing hair, longer hair, and/or thicker hair. In some embodiments, the candidate is a subject who wishes to increase hair pigmentation. In some embodiments, the subject is not affected by a condition of excessive hair loss.

As used herein, the terms “patient” and “subject” are used interchangeably.

(I) Scarring Alopecia

In some embodiments, the subject has scarring (cicatricial) alopecia. Forms of cicatricial alopecia that may be treated in accordance with the methods described herein include primary cicatricial alopecia (PCA) and secondary cicatricial alopecia. Primary cicatricial alopecias that may be treated in accordance with the methods described herein include lymphocyte-mediated PCAs, such as lichen planopilaris (LPP), frontal fibrosing alopecia (FFA), central centrifugal cicatricial alopecia (CCCA), and pseudopelade (Brocq); neutrophil-mediated PCAs, such as folliculitis decalvans and tufted folliculitis; and PCAs involving a mixed inflammatory infiltrate, such as occurs in dissecting cellulitis and folliculitis keloidalis.

In some embodiments, in a candidate subject for treatment, the area affected by the scarring alopecia is no longer increasing. In some embodiments, in a candidate subject for treatment, hair loss has in the affected area has ceased. In some embodiments, a candidate subject for treatment is clinically quiescent with respect to the inflammatory activity that may be associated with the condition. In one embodiment with respect to a subject having a lymphocyte-mediated PCA, inflammation is measured as the number of T lymphocytes and/or T lymphocyte subsets as detected in lesional skin, e.g., by immunoperoxidase cell surface staining using monoclonal antibodies. In another embodiment with respect to a subject having a lymphocyte-mediated PCA, lymphocytic inflammation (which may be found along with necrotic keratinocytes) is detected by histologic examination of the scalp. In another embodiment, direct immunofluorescence staining techniques are employed to detect antibody deposits in the affected tissue. In certain embodiments, clinical evaluation of the scalp is performed to determine clinical quiescence of the inflammation. Symptoms of itching, burning, pain, or tenderness usually signal ongoing activity. Signs of scalp inflammation include redness, scaling, and pustules. In certain embodiments, a scalp biopsy can be performed to demonstrate active inflammation or its absence. In certain embodiments, a hair “pull test” is performed to identify areas of active disease in which follicles are easily pulled out, and thus, inflammation is still ongoing. The pulled hairs can be mounted on a slide and the hair bulbs are viewed with a microscope to determine how many are growing hairs and how many are resting hairs. In addition, if pustules are present, cultures may be performed to identify which microbes, if any, may be contributing to the inflammation. In certain embodiments, a subject is clinically quiescent if hairs cannot be easily pulled out, if itching, burning, pain, tenderness, redness, scaling, and / or pustules are absent from the affected area.

In some embodiments, a method described herein is used to enhance hair growth in a patient with scarring alopecia. In some embodiments, the patient has a secondary cicatricial alopecia. In some embodiments, the patient has a form of primary cicatricial alopecia, such as lymphocyte-mediated PCAs, such as lichen planopilaris (LPP), frontal fibrosing alopecia (FFA), central centrifugal cicatricial alopecia (CCCA), and pseudopelade (Brocq); neutrophil-mediated PCAs, such as folliculitis decalvans and tufted folliculitis; and PCAs involving a mixed inflammatory infiltrate, such as occurs in dissecting cellulitis and folliculitis keloidalis.

Cicatricial alopecias affect both men and women, most commonly adults, although all ages may be affected. In general, they are rare. There have been a few reports of cicatricial alopecia occurring in a family. However, the majority of patients with cicatricial alopecia have no family history of a similar condition. Lichen planopilaris may affect middle-aged women most commonly. Central centrifugal alopecia may affect black women most commonly. Frontal fibrosing alopecia is seen most commonly in post-menopausal women. Thus, in certain embodiments, in addition to the subjects described herein, a candidate subject for treatment for scarring alopecia is a black woman (e.g., of African-American descent), a middle-aged woman, or a post-menopausal woman.

In a specific embodiment, the invention provides a method for enhancing hair growth in a patient with lichen planopilaris comprising controlled integumental perturbation using a fractional ablative laser, followed by twice daily topical administration of a pharmaceutical composition comprising valproic acid or a pharmaceutically acceptable salt thereof for 14 days. In certain embodiments, administration of a pharmaceutical composition comprising valproic acid or a pharmaceutically acceptable salt is begun on the same day as the laser treatment. In certain embodiments, administration of a pharmaceutical composition comprising valproic acid or a pharmaceutically acceptable salt is commenced on the same day as the integumental perturbation and is continued once, twice, three times, four times, or five times daily for 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

In another specific embodiment, the invention provides a method for enhancing hair growth in a patient with frontal fibrosing alopecia comprising controlled integumental perturbation using a fractional ablative laser, followed by twice daily topical administration of a pharmaceutical composition comprising valproic acid or a pharmaceutically acceptable salt for 14 days. In certain embodiments, administration of a pharmaceutical composition comprising valproic acid or a pharmaceutically acceptable salt is begun on the same day as the laser treatment. In certain embodiments, administration of a pharmaceutical composition comprising valproic acid or a pharmaceutically acceptable salt is commenced on the same day as the integumental perturbation and is continued once, twice, three times, four times, or five times daily for 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or 21 days.

For example, in some embodiments, an affected area of the skin is transplanted with hair follicles from an unaffected area. In some embodiments, surgical techniques for replacing tissue comprising scarred hair follicles with tissue from another area of the skin (e.g., scalp) comprising unaffected hair follicles are used. Surgical treatment for cosmetic benefit is an option in, for example, some cases after the disease has been inactive for one to two or more years. Hair restoration surgery or scalp reduction may be considered in these instances. Thus, in some embodiments, the integumental perturbation is a form of scar revision, such as skin graft, serial expansion of surrounding skin, or laser treatment. In some embodiments, the integumental perturbation is a form of scar re-excision with subsequent healing by primary intention, treatment with steroids (e.g., corticosteroid injection), silicone scar treatments (e.g., dimethicone silicone gel or silicone sheeting), use of porcine fillers or other cosmetic fillers (e.g., inserted under atrophic scars), ribosomal 6 kinase (RSK) antagonists, antagonists of pro-inflammatory cytokines, such as TGFβ2 or TNF, osteopontin antagonists, the use of pressure garments, needling, dermabrasion, collagen injections, low-dose radiotherapy, or vitamins (e.g., vitamin E or vitamin C or its esters).

### (II) Androgenetic Alopecia

Both males and females develop diffuse hair loss in the frontal/parietal scalp called “thinning,” which begins between 12 and 40 years of age. In females, thinning is known as “Female Pattern Hair Loss (FPHL)” and is caused or exacerbated by androgens. (Price VH, 2003, J Investig Dermatol Symp Proc. 8(1):24-7, Androgenetic alopecia in women).

### (III) Male Pattern Hair Loss (MPHL)

After puberty, males begin to lose the scalp hair over the vertex, crown and frontal/parietal areas in a relatively characteristic pattern that is a continuum (described by Hamilton Norwood scale). The loss of scalp hair in men is called MPHL and is known to be a process driven by the androgen, dihydrotestosterone (DHT), which can be inhibited and to some extent reversed by finasteride which inhibits the conversion of testosterone to DHT. Minoxidil can also delay or reverse MPHL.

### (IV) Aging

Aging of humans results in programmed hair patterning. Diffuse hair loss, including thinning of the occipital scalp occurs in aging. This can either be an extension of androgenetic alopecia (MPHL or FPHL) from the earlier years or even start in the latter decades of life when amounts of testosterone and DHT in the body are decreasing.

It is believed that hair loss in postmenopausal women is related to the loss of estrogens (and/or a decrease in the estrogen/androgen ratio). Accordingly, in some embodiments, the combination treatments disclosed herein for age-related hair loss comprise a combination of treatment with one or more hair growth-promoting agents and estrogen replacement therapy or androgen inhibition therapy.

Aging also results in change of follicle cycle control. In males, eyebrows grow longer and nares hair grow longer suggesting that the lengths of telogen and anagen are no longer regulated as closely. In other words, with aging there is a loss of the function of suppressing terminal hair growth.

### (V) Hair Color Changes

Hair color changes in both males and females becoming progressively grayer (mixture of gray hair; white hair and black hair) and whiter. Color change is patterned, since scalp hair changes earlier than body beard hair or body hair. Beard hair may also change color in a pattern that follow a moustache line, before ultimately turning uniformly gray (typically a mixture of white and black hair). This is due to decreased melanin content in the hair shaft (supplied by melanocytes associated with hair follicles).

(VI) Factors that Regulate Sex Hormone Sensitivity of Hair Follicle Cells

Cytokines regulate the activity of Dermal Papillae, which is believed to be the target of androgen regulation of hair growth. Interleukin-1 alpha decreases responses to androgen in cultured dermal papilla cells (Boivin et al., 2006, Exp Dermatol. 15:784-793). TGF-betal may mediate androgen-induced hair growth suppression, since in culture, human dermal papilla cells (DPCs) from androgenetic alopecia (AGA) subjects that transiently expressing androgen receptor were co-cultured with keratinocytes (KCs), and secreted TGF-betal that inhibited KC growth (Inui et al., 2003, J Investig Dermatol Symp Proc. 8:69-71).

In certain embodiments, adjuvants and/or other stimulators of local cytokines are used in conjunction with the treatment with one or more hair growth-promoting agents. Without being bound by any theory, one rationale for administering adjuvants and/or other stimulators of local cytokines in conjunction with the treatment with one or more hair growth-promoting agents is that the production of local cytokines may induce changes in the follicle cell cycle and recruit new FSCs to follicles.

Melatonin is a protein hormone secreted by the pineal gland modulates hair growth, pigmentation and/or molting in many species. Human scalp hair follicles in anagen are important sites of extra-pineal melatonin synthesis. Melatonin may also regulate hair Follicle Cycle control, since it inhibits estrogen receptor-alpha expression (Fischer et al., 2008, Pineal Res. 44:1-15). These treatments can be administered in combination with the methods described herein.

### (VII) Treatments for Delaying or Reversing Hair Patterning

Given the regulation of human hair patterning by sex steroids, it is believed that humans evolved hair patterning to provide social signals in interactions such as mating and dominance. However, current fashion motivates many men to prevent, delay or reverse male MPHL.

Women also suffer from hair thinning and hair loss due to a variety of factors; for example, certain conditions, such as, e.g., polycystic ovary, result in male-pattern facial and body hair on females, which motivates them to remove or reduce hair. Many women also desire the prevention, delay or reversal of “female-pattern baldness,” which may result from a variety of factors, for example, the aging process.

### 6.5 ADDITIONAL AGENTS

The methods described herein (e.g., methods comprising integumental perturbation in combination with administration of valproic acid or a pharmaceutically acceptable salt thereof), alone or in combination administration of additional agents or active ingredients, for example hair growth-promoting agents, and optionally in combination with the treatments described herein. Additional agents include, in some embodiments, hair growth-promoting agents.

In some embodiments, a hair growth-promoting agent described herein promotes hair follicle development and growth, resulting in the transition of vellus hair on an area of the skin to non-vellus, e.g., intermediate or terminal, hair. In some embodiments, a hair growth-promoting agent described herein acts synergistically with the integumental perturbation method to promote hair growth. The effect that each treatment offers could be an additive or synergistic improvement, or a combination of two different biologically defined effects, to achieve the desired end result.

In some embodiments, the hair growth-promoting agent is a treatment that promotes hair growth and/or treats a disease or condition associated with excessive hair loss. Any treatment that promotes hair growth and/or treats a disease or condition associated with excessive hair loss that is known in the art or yet to be developed is contemplated for use in accordance with these embodiments. As used herein, the term “hair growth-promoting agent” refers to any agent that promotes hair growth or hair thickness, or is intended for such purpose, and/or treats a disease or condition associated with hair loss, or is intended for such purpose. In some embodiments, the hair growth-promoting agent is an agent that promotes, or is intended to promote, the transition of vellus hair to non-vellus hair. In some embodiments, the hair growth-promoting agent is an agent that promotes, or is intended to promote, the transition of vellus hair to terminal hair. In some embodiments, the hair growth-promoting agent increases vellus hair growth. In some embodiments, the hair growth-promoting agent increases non-vellus hair growth. In some embodiments, the hair growth-promoting agent increases terminal hair growth. In some embodiments, the hair growth-promoting agent increases the ratio of non-vellus-to-vellus hair on an area of skin of a subject. In some embodiments, the hair growth-promoting agent increases the ratio of terminal-to-vellus hair on an area of skin of a subject. In some embodiments, the hair growth-promoting agent maintains nono-vellus hair growth. In some embodiments, the hair growth-promoting agent maintains terminal hair growth, i.e. helps prevent miniaturization of terminal hairs. In some embodiments, the hair growth-promoting agent increases the number of anagen hairs or increases anagen hair growth. In some embodiments, the hair growth-promoting agent increases the ratio of anagen-to-telogen hair on an area of skin of a subject.

In some embodiments, the hair growth-promoting agent treatment comprises treatment with one or more channel openers (e.g., potassium channel opener, e.g., an ATP-sensitive potassium channel (KATP opener), or an activator of such a channel), such as, e.g., minoxidil (e.g., marketed as Rogaine or Regaine), diazoxide, or phenytoin.

In some embodiments, the hair growth-promoting agent treatment comprises treatment with one or more 5α-reductase inhibitors. Non-limiting examples of 5α-reductase inhibitors include finasteride, dutasteride (e.g., Avodart), turosteride, bexlosteride, izonsteride, epristeride, epigallocatechin, MK-386, azelaic acid, FCE 28260, and SKF 105,111. Commonly used dosage forms of finasteride that may be used in such treatments are, for example, oral finasteride at 1 mg/day. See, e.g., Physicians' Desk Reference, 2009, 63rd ed., Montvale, N.J.: Physicians' Desk Reference Inc., entries for Propecia® and Proscar® at pages 2095-2099 and 2102-2106, respectively, which are incorporated herein by reference in their entireties.

In some embodiments, the hair growth-promoting agent treatment comprises treatment with one or more antiandrogens, such as, e.g., finasteride (e.g., marketed as Propecia or Proscar), ketoconazole, fluconazole, spironolactone, flutamide, diazoxide, 17-alpha-hydroxyprogesterone, 11-alpha-hydroxyprogesterone, ketoconazole, RU58841, dutasteride (marketed as Avodart), fluridil, or QLT-7704, an antiandrogen oligonucleotide, or others described in Poulos & Mirmirani, 2005, Expert Opin. Investig. Drugs 14:177-184, the contents of which is incorporated herein by reference.

In some embodiments, the hair growth-promoting agent treatment comprises treatment with one or more prostaglandin F2a analogs, prostaglandin analogs, or prostaglandins. Non-limiting examples of prostaglandin F2a analogs include bimatoprost (e.g., Latisse, Lumigan), latanoprost (trade name Xalatan), travoprost (trade name Travatan), tafluprost, unoprostone, dinoprost (trade name Prostin F2 Alpha), AS604872, BOL303259X, PF3187207, carboprost (trade name Hemabate). For exemplary prostaglandin F2a analogs, as well as formulations, dosages, and treatment regimens, for use in accordance with the methods described herein, see, e.g., U.S. Pat. Nos. 8,017,655, 5,688,819, 6,403,649, 5,510,383, 5,631,287, 5,849,792, 5,889,052, 6,011,062, 7,163,959, 5,296,504, 5,422,368, 6,429,226, and 6,946,120, the entire contents of each of which is incorporated herein by reference in its entirety. See also, with respect to latanoprost, Uno et al., 2002, Acta Derm Venereol 82:7-12, the contents of which is incorporated herein by reference in its entirety.

In some embodiments, treatment optionally comprises treatment with one or more of the following hair growth-promoting agents: kopexil (for example, the product KeraniqueTM) CaC12, botilinum toxin A, adenosine, ketoconazole, DoxoRx, Docetaxel, FK506, GP11046, GP11511, LGD 1331, ICX-TRC, MTS-01, NEOSH101, HYG-102440, HYG-410, HYG-420, HYG-430, HYG-440, spironolactone, CB-03-01, RK-023, Abatacept, Viviscal®, MorrF, ASC-J9, NP-619, AS101, Metron-F-1, PSK 3841, Targretin (e.g., 1% gel), MedinGel, PF3187207, BOL303259X, AS604872, THG11331, PF-277343, PF-3004459, Raptiva, caffeine, an coffee. In some embodiments, the hair growth-promoting agent treatment comprises drugs for alopecia being developed by SWITCH Biotech LLC.

In some embodiments, the hair growth-promoting agent treatment comprises treatment with one or more of the following: herbs (such as, e.g., saw palmetto, glycine soja, Panax ginseng, Castanea Sativa, Arnica Montana, Hedera Helix Geranium Maculatum), triamcinolone acetonide (e.g., suspension of 2.5 to 5 mg/ml for injection), a topical irritant (e.g., anthralin) or sensitizer (e.g., squaric acid dibutyl ester [SADBE] or diphenyl cyclopropenone [DPCP]), clomipramine, unsaturated fatty acids (e.g., gamma linolenic acid), a fatty acid derivative, thickeners (such as, e.g., carbomer, glycol distearate, cetearyl alcohol), a hair loss concealer, niacin, nicotinate esters and salts, adenosine, and methionine. In some embodiments, the hair growth-promoting agent treatment comprises treatment with nitroxide spin labels (e.g., TEMPO and TEMPOL). See U.S. Pat. No. 5,714,482, which is incorporated herein by reference.

In some embodiments, the hair growth-promoting agent treatment comprises treatment with an androgen receptor inhibitor, which have been shown to be useful for stimulating scalp hair growth (Hu LY, et al., 2007, Bioorg Med Chem Lett. 2007 17:5983-5988).

In some embodiments, the hair growth-promoting agent treatment comprises treatment with a copper peptide(s), preferably applied topically, or another compound with superoxide dismutation activity. In some embodiments, the hair growth-promoting agent treatment comprises treatment with an agent that increases nitric oxide production (e.g., arginine, citrulline, nitroglycerin, amyl nitrite, or sildenafil (Viagra)). In preferred embodiments, such compounds are administered further in combination with a catalase or catalase mimetic, or other antioxidant or free radical scavenger.

In some embodiments, the hair growth-promoting agent treatment comprises treatment with a compound that mobilizes bone marrow-derived stem cells (e.g., growth factors such as G-CSF and/or chemical agents such as plerixafor (Mozobil®)); and/or that regulates the differentiation of these stem cells into gender-specific specialized human hair follicles (e.g., using agents such as finasteride, fluconazole, spironolactone, flutamide, diazoxide, 11-alpha-hydroxyprogesterone, ketoconazole, RU58841, dutasteride, fluridil, or QLT-7704, an antiandrogen oligonucleotide, cyoctol, topical progesterone, topical estrogen, cyproterone acetate, ru58841, combination 5a-reductase inhibitors, oral contraceptive pills, and others in Poulos & Mirmirani, 2005, Expert Opin. Investig. Drugs 14:177-184, incorporated herein by reference, or any other antiestrogen, an estrogen, or estrogen-like drug (alone or in combination with agents that increase stem cell plasticity; e.g., such as valproate), etc., known in the art), that can result in, e.g., the appearance of specialized follicles having features that are different from natural follicles in the target location of skin.

In some embodiments, the hair growth-promoting agent treatment comprises treatment with one or more agents that counteract age-related hair thinning and/or hair follicle cell senescence (also referred to herein as “anti-senescence agents”) for example, anti-oxidants such as glutathione, ascorbic acid, tocopherol, uric acid, or polyphenol antioxidants); inhibitors of reactive oxygen species (ROS) generation, such as superoxide dismutase inhibitors; stimulators of ROS breakdown, such as selenium; mTOR inhibitors, such as rapamycin; or sirtuins or activators thereof, such as resveratrol, or other SIRT1, SIRT3 activators, or nicotinamide inhibitors.

In some embodiments, the hair growth-promoting agent treatment comprises treatment with one or more agents that induce an immune response or cause inflammation, such as, e.g., tetanus toxoid, topical non-specific irritants (anthralin), or sensitizers (squaric acid dibutyl ester [SADBE] and diphenyl cyclopropenone [DPCP]). While not intending to be bound by any theory, it is thought that by contacting these agents to the skin, lymphocytes and hair follicle stem cells may be recruited to skin. In some embodiments, the hair growth-promoting agent treatment comprises treatment with a chemical or mechanical (such as those discussed infra) treatment that induces an inflammatory process in the skin. While not intending to be bound by any theory, inducing inflammation in the site where hair growth is desired helps to recruit stem cells to the tissues that drive the formation of new follicles.

In some embodiments, the hair growth-promoting agent treatment comprises treatment with an antiapoptotic compound. In one embodiment, the antiapoptotic compound is not a Wnt or a Wnt agonist.

In some embodiments, the hair growth-promoting agent treatment comprises treatment with stem cell therapy, hair cloning, hair transplantation, scalp massage, a skin graft, hair plugs, follicular unit extraction, or any surgical procedure aimed at hair restoration.

In certain embodiments, a hair growth-promoting agent described herein may be used at a dosage or in a range of dosages known in the art for that agent (e.g., as made available on a package insert or in the Physicians' Desk Reference). In other embodiments the regular dosage of the hair growth-promoting agent is adjusted to optimize a combination treatment (e.g., integumental perturbation or treatment with another active ingredient) described herein. For example, the regular dosage may be increased or decreased as directed by the physician. For example, a lower dosage may be used over a shorter duration owing to the synergistic effect of combination with another treatment described herein.

In certain embodiments, the hair growth-promoting agent may be used in its commercially available form. In other embodiments, the form of the hair growth-promoting agent is adjusted to optimize a combination treatment (e.g., integumental perturbation or treatment with another active ingredient) described herein. In a particular embodiment, the hair growth-promoting agent is formulated as a different salt form than that which is commercially available. In a particular embodiment, the hair growth-promoting agent is formulated for topical administration, e.g., by incorporation into a pharmaceutical composition for treatment described in Section 6.1.3 infra.

In some embodiments, the hair growth-promoting agent enhances conversion of vellus hair to non-vellus hair. In a particular embodiment, the hair growth-promoting agent enhances conversion of vellus hair to terminal hair. Exemplary hair growth-promoting agents that promote conversion of vellus to non-vellus hair that may be used in accordance with these embodiments are prostaglandin F2α analogs (in one aspect, latanoprost), minoxidil, etc. In some embodiments, the hair growth-promoting agent enhances conversion of telogen hair to anagen hair. In a particular embodiment, the hair growth-promoting agent enhances conversion of telogen hair to anagen hair. Exemplary hair growth-promoting agents that promote conversion of telogen to anagen hair that may be used in accordance with these embodiments are prostaglandin F2α analogs (in one aspect, latanoprost), minoxidil, etc.

In some embodiments, the hair growth-promoting agent treatment comprises treatment with an antiandrogen (e.g., a 5α-reductase inhibitor) and a channel opener (e.g., minoxidil). In one such embodiment, a 5α-reductase inhibitor is administered in combination with minoxidil. In one such embodiment, finasteride is administered in combination with minoxidil. In some embodiments, the hair growth-promoting agent treatment comprises treatment with a prostaglandin F2α or prostamide analog (e.g., latanoprost, bimatoprost, etc.) in combination with a channel opener (e.g., minoxidil). In one such embodiment, a prostaglandin F2α or prostamide analog is administered in combination with minoxidil. In one such embodiment, latanoprost is administered in combination with minoxidil. In another such embodiment, bimatoprost is administered in combination with minoxidil.

In some embodiments, a treatment described herein for promoting hair growth in a female subject does not comprise finasteride or ketoconazole. In some embodiments, a treatment described herein for promoting hair growth in a pregnant female subject is not finasteride or ketoconazole.

In some embodiments a treatment described herein for promoting hair growth does not comprise minoxidil. In some embodiments a treatment described herein for promoting hair growth does not comprise finasteride. In some embodiments a treatment described herein for promoting hair growth does not comprise dutasteride. In some embodiments a treatment described herein for promoting hair growth does not comprise fluridil. In some embodiments a treatment described herein for promoting hair growth does not comprise spironolactone. In some embodiments a treatment described herein for promoting hair growth does not comprise cyproterone acetate. In some embodiments a treatment described herein for promoting hair growth does not comprise bicalutamide. In some embodiments a treatment described herein for promoting hair growth does not comprise flutamide. In some embodiments a treatment described herein for promoting hair growth does not comprise nilutamide. In some embodiments, a treatment described herein for promoting hair growth does not comprise an inhibitor of an androgen receptor. In some embodiments a treatment described herein for promoting hair growth does not comprise an androgen antagonist. In some embodiments a treatment described herein for promoting hair growth does not comprise an anti-androgen.

### 6.6 KITS

In an example, the pharmaceutical compositions described in the methods described herein may each be packaged and sold separately, packaged separately and sold in another packaging together, or packaged and sold together.

For example, the pharmaceutical compositions described above may be sold in a package including a pharmaceutical composition (e.g., a pharmaceutical composition comprising valproic acid or a pharmaceutically acceptable salt) and a needling device. The package may additionally comprise an adaptor sheath with a rectangular needing array, and a charging station. Also, the needling device may be sold without the charging station. Also, the needling device may be sold with a one or more additional needling adaptors or sheaths having a different needling array such as a circular needling array for providing a precision tip.

An embodiment of needling devices and components as described above may be sold with a fluid or drug applicator. The drug applicator may include the applicator device, one or more massage heads, one or more massage cartridges, and an applicator charging station.

The needling device and applicator can be sold together as a system with a needling device, a needling sheath adaptor, a fluid applicator, a fluid cartridge, one or more charging stations, and a software or a downloadable mobile app for controlling each of the devices together and/or separately for procedures involving needling, massaging, and fluid dispensing cycles, separately or in combination.

It will be appreciated by those skilled in the art that changes could be made to the embodiments described above without departing from the broad inventive concept thereof. It is understood, therefore, that the invention disclosed herein is not limited to the particular embodiments disclosed, but it is intended to cover modifications within the spirit and scope of the present invention as defined by the appended claims.

### 6.7 GLOSSARY OF TERMS FOR HAIR AND DISORDERS OF HAIR GROWTH

The following terms are used herein consistently with their art-accepted meanings summarized below.

Alopecia: Abnormal hair loss:

Alopecia areata: Hair loss in patches, thought to be caused by an autoimmune response to hair follicles in the anagen stage; extensive forms of the disorder are called alopecia areata totalis (hair loss over the entire scalp) and alopecia areata universalis (hair loss over the entire body).

Anagen: Growth stage of the hair-Follicle Cycle.

Anagen effluvium: Abrupt shedding of hair caused by interruption of active hair-follicle growth (e.g., in patients undergoing chemotherapy).

Androgenetic alopecia (AGA): Baldness caused by miniaturization of genetically predisposed follicles in the MPHL pattern (frontal recession and thinning at the vertex) or the FPHL pattern (loss of hair primarily over the crown, with sparing of frontal hair).

Bulb: Lowermost portion of the hair follicle, including the dermal papilla (also known as the follicular papilla), containing rapidly proliferating matrix cells that produce the hair.

Bulge: Portion of the outer-root sheath of the hair follicle, located at the region of the insertion of the arrector pili muscle; thought to contain epithelial stem cells responsible for regenerating follicles in the anagen stage.

Catagen: Stage of the hair cycle characterized by regression and involution of the follicle.

Club hair: Fully keratinized, dead hair—the final product of a follicle in the telogen stage; 50 to 150 club hairs are shed daily from a normal scalp.

Female Pattern Hair Loss (FPHL): form of gender specific hair patterning in females (also sometimes referred to as female pattern alopecia).

Follicle cycle: Hair growth in each follicle occurs in a cycle that includes the following phases: anagen (growth phase), catagen (involuting/regressing stage), telogen (the quiescent phase), exogen (shedding phase), kenogen, and re-entry into anagen.

Kenogen: Latent phase of hair cycle after hair shaft has been shed and growth is suspended in follicle.

Hirsutism: Excessive hair growth in androgen-dependent areas in women.

Hypertrichosis: Excessive hair growth (usually diffuse) beyond that considered normal according to age, race, sex, and skin region.

Integumental: Pertaining to the integumentary system, which comprises the skin (epidermis, dermis, hypodermis (or subcutanea)) and all cells contained therein regardless of origin, and its appendages (including, e.g., hair and nails).

Intermediate hair: Hair shafts are typically 30 p.m to 60 p.m in diameter.

Lanugo hair: Fine hair on the body of the fetus, usually shed in utero or within weeks after birth.

Male Pattern Hair Loss (MPHL): form of gender specific hair patterning in men (also sometimes referred to as male pattern alopecia).

Miniaturization: Primary pathological process in androgenetic alopecia, resulting in conversion of large (terminal) hairs into small (vellus) hairs.

NL (Neogenic-Like) follicular structure: In certain embodiments, an unattached primitive follicular structure, with only one of the following “small” traits: shaft, sebaceous gland, or pore. Dermal channel is absent or inconclusive. Further subcategories of NL include: NL with DP (dermal papilla)/active, NL with DP/inactive, NL without DP/active, and NL without DP/inactive.

Non-vellus hair: Non-vellus hair is any hair that is not vellus hair. Non-vellus hair inter alia includes terminal hair and intermediate hair.

Non-vellusPEL (Pre-Existing-Like) follicular structure: In certain embodiments, an unattached primitive follicular structure, with one or more of the following “large” traits or two or more of the following “small” traits: shaft, sebaceous gland, or pore. Dermal channel is present. Further subcategories of PEL include: PEL with DP (dermal papilla)/active, PEL with DP/inactive, PEL without DP/active, and PEL without DP/inactive.

PELA (Pre-Existing-Like, Attached) follicular structure: In certain embodiments, a primitive follicular structure that is attached to larger, mature, pilosebaceous unit that extends to the epidermis.

Permanent alopecia: Caused by destruction of hair follicles as a result of inflammation, trauma, fibrosis, or unknown causes; examples include lichen planopilaris and discoid lupus erythematosus. Includes diseases referred to as scarring alopecia.

Telogen: Resting stage of the hair cycle; club hair is the final product and is eventually shed.

Telogen effluvium: Excessive shedding of hair caused by an increased proportion of follicles entering the telogen stage; common causes include drugs and fever.

Terminal hair: Large, usually pigmented hairs on scalp and body. Hair shaft diameters are typically 60 μm or greater.

Vellus hair: Very short, often nonpigmented hairs (e.g., those found diffusely over nonbeard area of face and bald scalp as a result of miniaturization of terminal hairs). In certain embodiments, as used herein, a “vellus” hair is a hair that is less than 2 mm in length and less than 30 μm in diameter. In certain embodiments, as used herein, a “vellus” hair is a hair that is determined histologically as having a hair shaft diameter of less than 30 μm and not exceeding the thickness of its surrounding internal root sheath.

### 4.8 EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The Examples do not in any way limit the disclosure.

### Example 1

### An Investigation of the Effectiveness of Novel Compounds in Increasing Neofollicle Germ Area Following a Full Thickness Excision (FTE) Procedure in Mice

A study was conducted to assess the degree to which valproic acid could stimulate growth of new hair follicles by in mice when applied after a full thickness excision (FTE) procedure. FTE creates a zone of hair follicles that are 100% newly formed and removes noise from other clouding factors (e.g., migration of pre-existing follicles into the wound) (e.g., as described in Ito et. al., Nature, May 2007, “Wnt-dependent de novo hair follicle regeneration in adult mouse skin after wounding”, the contents of which is herein incorporated by reference in its entirety). The study was conducted with 2 arms (1) vehicle control and (2) 8.3% valproic acid; n=21 mice per arm). Valproic acid or placebo was applied twice daily for 4 days following scab detachment. The resulting data was analyzed and compared to a vehicle compound control cohort.

Surgery

Day-12 C57BL6/J female mice pups (Jackson Labs) were maintained in cages and fed a high fat diet until day of surgery. At 21 days of age, the female mice pups were weighed, and all pups >7g were considered eligible for FTE surgery. Mice selected for the study were injected with buprenorphine (BUP, 0.05 mg/kg). After 1 hour, the mice were injected with ketamine (70 mg/kg)-xylazine (8 mg/kg), assigned a Study ID, and weight was recorded. Back hair was shaved an a standardized 1.5×1.5 cm area was traced onto on the rear dorsum of each animal and used as a guide for surgical excision of the epidermis and dermis, and the surgery site was sterilized with 70% ethanol to prevent infections. A pair of curved tipped forceps was used to pinch the skin upwards along the traced perimeter. Blunt-tip or curved-tip scissors were used to cut the animal skin along the box perimeter, and the complete dermis and epidermis were removed. After completion of FTE, mice were placed pre-warmed cages with access to regular food, strawberry Jello, and water and an oral rehydrator (i.e. Strawberry Prang) and allowed to recover for two days. BUP pain medication (0.05 mg/kg) was added to dish of Jello for both AM and PM doses (approx. 10am and 5pm) on Day 1 and Day 2 post-FTE. Regular food was replaced as needed. Scabs were observed close to the the wound within 1-2 days post-FTE, which reached significant detachment (80% of full scab detached) between 11-18 days post-FTE.

The area was considered detached if there is no remaining bleeding or red spots in the detached area.

Upon loss of dorsal scab, animals were randomized into the dosing regimen. Valproic acid (8.3% valproic acid in 50% (vol/vol) ethanol, 30% water, and 20% propylene glycol) or vehicle alone (50% (vol/vol) ethanol, 30% water, and 20% propylene glycol) was applied topically to the recently healed wound area using a pipette. A dose volume of 20 uL was given twice daily and applied evenly across the site for a duration of 4 days._Upon completion of the dosing regimen (SD1-SD4) each animal was euthanized via CO2 (SD5) and confocal imaging was performed.

Confocal Imaging

The degree of neofollicle germ formation was measured in the wound area, using imaging by confocal microscopy (confocal scanning laser microscope (CSLM) (VivaScope 1500, Lucid Inc., Rochester, N.Y., USA) and counting the number of early hair follicles developing underneath the surface of the skin.

A small drop of index matching fluid (STE Oil Crystal Plus 500FG) was placed on the surface of the wound to match the index of refraction of the stratum corneum (SC) in order to increase the visibility of hair follicles. A metallic tissue ring holder was placed together with a disposable 30 mm medical-grade adhesive polycarbonate window (Lucid Inc.) onto the wound and surrounding fur to create a secure well for holding the water-based immersion medium. A drop of ultrasound transmission gel immersion media (Aquasonics, Parker Laboratories Inc., Fairfield, N.J., USA) was applied inside the tissue ring on the surface of the window. The CSLM was attached magnetically to the metallic tissue holder affixed to the animal. Live scan was used to identify the dermal/epidermal junction and establish this level as Z=0. Images were set at Z=−10 μ, 0 μ, 10 μ, 20 μ, & 30 μ with one imaging window per mouse. A total of 64 images were combined to track germs throughout the dermis across 5 layers. The image set was saved to a computer as a bitmap image.

Confocal Analysis

The total wound area was outlined in ImageJ (image processing software) for each subject image, from which the total wound size (mm²) was calculated. Within the wound area, the boundary of the neofollicle germ cluster was outlined in the software (mm²) and identified as the germ forming region (GFR). From the full surface bitmap image, the number of neofollicle germs in the GFR were counted, scored, and recorded.

Tissue Collection & Storage

After completion of the confocal imaging, a 1 mm² section of the wound area was excised from the animal and placed into 3m1 of paraformaldehyde for 48 hours. Once the tissue is fixed, sections were placed into medium (Tissue Tek OCT Cryo Gel), and container (Tissue Tek Cryo Mold Intermediate) and frozen in liquid nitrogen. Alkaline phosphatase (AP) stained sections were used as an additional means to count and score the number of neofollicle germs in the GFR.

Scoring

Prior to unblinding, counting was conducted independently by at least 2 separate persons counting the number of neofollicles in confocal images and AP stained sections. An adjudication process was followed to assign samples to three categories as shown in Table 1. Scoring and analysis results are shown in Table 2. Overall, a strong trend indicating increased numbers of neofollicles was observed upon FTE+VPA treatment as compared to FTE +vehicle treated controls. Upon combination of primary 1 and primary 2 category counts, the mean count for VPA treated FTEs was 37.1 as compared to 26.3 for vehicle control treated FTEs (P(T<=t) one-tail: 0.066).

TABLE 1
Neofollicle Scoring Categories and Method

| Scoring catagories | Description | Scoring method |
|---|---|---|
| Primary 1 IF (Valproic | Confocal images of high | Use confocal count as determined by |
| Acid: n = 7; Vehicle | quality, strong confidence in | primary counter |
| control n = 9) | count; AP not performed or | |
| | was of lower confidence than | |
| | confocal | |
| | Confocal images of sufficient | Use confocal count averaged between |
| | quality to gain strong confidence | primary and secondary counters |
| | in count | |
| | AP not performed or was of | |
| | lower confidence than | |
| | confocal | |
| | Confocal and AP images of | AP or confocal selected for count |
| | sufficient quality to gain | (whichever count was higher) |
| | strong confidence in count | |
| | Numbers aligned between | |
| | confocal and AP | |
| Primary 2 IF (Valproic | Confocal and AP present,either | If AP of best quality, AP count was |
| Acid: n = 8; Vehicle | confocal and/or AP of sufficient | selected otherwise confocal counts |
| control n = 12) | quality to gain moderate | were used; count was averaged between |
| | confidence in count | primary and secondary counters |
| | Confocal of sufficient quality | Confocal count was averaged between |
| | to gain moderate confidence in | primary and secondary counters |
| | count (limited areas were present | |
| | which could not be scored (“blind | |
| | spots”); AP not performed or was | |
| | of lower confidence than confocal | |
| Secondary IF (Valproic | Confocal and AP available, either | If AP of best quality, AP count was |
| Acid: n = 4; Vehicle | were of sufficient quality to | selected, otherwise confocal image |
| control n = 0) | gain low confidence in count | was used for scoring |
| | | Average of primary and secondary |
| | | count was used |
| Excluded IF (Valproic | Confocal of low quality; if | Exclusion from dataset |
| Acid: n = 2; Vehicle | available, AP was of low quality; | |
| control n = 0) | large number of artifacts or | |
| | significant difference between | |
| | confocal and AP counts | |

TABLE 2
Scoring Results

| Analyses | | Randomization | | |
|---|---|---|---|---|
| Group | n | Group | Mean | SD |
| Primary 1 + | 15 | Valproic Acid | 37.1 | 19.77 |
| Primary 2 | 21 | Vehicle Control | 26.3 | 21.59 |

### Example 2

### An Investigation of the Effectiveness of Valproic Acid (VPA) in Increasing Neofollicle Germ Area Following a Full Thickness Excision (FTE) Procedure in Mice

A follow on study is conducted to assess the degree to which valproic acid could stimulate growth of new hair follicles by in mice upon full thickness excision (FTE), essentially as described above, except that the number of animals per arm is increased to account for the variability of the model.

The study is conducted with 2 arms ((1) vehicle (50% (vol/vol) ethanol, 30% water, and 20% propylene glycol and (2) 500 mM valproic acid (in vehicle). VPA is compared to a placebo arm (n=60 mice per arm). FTE is conducted as described above. As described above, VPA or placebo is applied twice daily for 4 days following scab detachment. Confocal analysis is conducted as described above. Both standard AP staining and fluorescent staining are performed on excised dermal and epidermal tissue, respectively, from each mouse following confocal imaging. The resulting data is analyzed and compared to the vehicle compound control

### Example 3

### Clinical Investigation of the Effectiveness of Valproic Acid in Increasing Hair Count Following Micro-needling Treatment

Using a device such as a micro-needling device described herein (e.g., Follica HFN Device , e.g., described at{0025} and elsewhere herein), the following micro-needling procedure is used. The method includes scalp assessment, global photography, and macrophotography procedures. First, the designated photography site (single, circular 1.9 cm² area) is identified and hair within targeted photography site is clipped to length of 1 mm. Next, a needlestick tattoo is created in the center of the targeted photography site for future orientation (apply pressure until any bleeding stops), the position of the tattoo is mapped, and the measurements are recorded. Using the tattoo as a reference point, the first baseline photograph without the contact plate is taken. Again using the tattoo as a reference point, the remaining baseline photography is performed with the contact plate.

The micro-needling procedure is done with a micro-needling device, such as Follica HFN Device, with a micro-needling penetration depth setting of 0.8mm. HFN device is glided over the affected skin area at a speed of 2cm/second, ensuring that the device is perpendicular to the surface of the skin at all times to ensure a consistent needle strike of 0.8mm micro-needling depth. Array of the device has 12 needles and the number of array oscillations per second is set at 120. The treatment width is 0.9 cm, and the translation speed is about 2.0 cm/s. As a result, 1600 needle strikes per cm\hat{ }2 are performed. The micro-needling device is glided across the affected area of the skin such that each pass across the area of skin aligns with the prior pass across the skin, without gaps of skin between the passes, in a “mow the lawn” approach. Each pass of the micro-needling device starts with a gliding start and a smooth retraction of the device from the skin area following each pass. The micro-needling device is applied to the skin area with consistent, light pressure, allowing the device to glide along the skin. The micro-needling device has a device alignment consisting of 6 rows of 2 microneedles.

### Example 4

### Protocol to Promote Hair Growth in Subjects with Androgenetic Alopecia

A randomized study is conducted to confirm the Follica HFN Device in conjunction with study drug (VPA, 500 mM or 8.3% VPA, topical) is safe and efficacious in adult males with hair loss, e.g., androgenetic alopecia (AGA). 60 subjects are randomized and then complete treatment across 12 weeks (total of 3, 6, or 12 treatments with the Follica HFN Device per randomization assignment, n=20 per arm). A final evaluation is conducted on Day 85 with no additional follow up required.

All study subjects receive a series of in-clinic procedures with the Follica HFN Device across the treatment period per their randomization assignment: Group 1: Follica HFN Device treatment every 4 weeks, 3 procedures total (Target days 1, 29, 57); Group 2: Follica HFN Device treatment every 2 weeks, 6 procedures total (Target days 1, 15, 29, 43, 57,71); Group 3: Follica HFN Device treatment every week, 12 procedures total (Target days 1, 8, 15, 22, 29, 36, 43, 50, 57, 64, 71, 78). Baseline (Day 0) and a subject's initial micro-needling procedure (Day 1) is performed on the same day.

Study drug is provided to each subject after their first wound closure assessment on Day 2. Subjects are instructed to apply study drugs per instructions throughout their participation in the study except for a rest period (minimum 24 hours) following any treatment with the device.

On Day 0, subjects are randomized to receive treatment with the Follica HFN Device on a weekly (12 treatments), bi-weekly (6 treatments), or monthly (3 treatments) basis for 12 weeks in a 1:1:1 ratio. Treatment of the vertex (including transitional areas) with the Follica HFN Device are initiated on Day 1 according to study procedure.

Subjects return to the clinic post-treatment with the HFN Device (24-36 hours) for an assessment of wound closure, to be independently confirmed by both subject and clinician. If the assessment does not confirm wound closure, an additional visit is scheduled 24-36 hours following.

Once closure is confirmed following initial treatment with the Follica HFN Device, subjects are provided VPA to be applied (see appropriate site of application, below) in accordance with the instructions provided. The very first application of study drug is performed in-clinic during the first post-treatment visit in which wound closure is confirmed. The study drug to be used includes VPA topical solution applied twice daily for 4 days.

At treatment Days 29 and 57, in-office visits across all arms also include repeat global photography of overall hair appearance. Subjects are contacted by phone or assessed during in-office visits on a weekly basis to confirm use of study drug, and report any concomitant medication usage or occurrence of any adverse events (AEs). The scalps are evaluated for signs of irritation or dermatologic conditions (e.g., erythema, edema, dryness, scaling) at Screening and Baseline visits as well as prior to any treatment with the Follica HFN Device. All AEs are coded and assessed for relation to the study device and study drug. A final report will include the number and type of each AE within each treatment group. At Day 85, an in-office visit includes repeat hair count analyses (macrophotography) and overall hair appearance (global photography), subject assessment of hair growth, and study-related AEs. Results are reported by randomization arm. Total planned duration of study participation and treatment per subject is 99 days, comprising a 14-day screening period (Day -14 to 0) and an 85-day period of treatment and follow-up (Day 1 to 85).

1. A method for promoting hair growth in a human subject, wherein the method comprises:
a. integumental perturbation of an area of the skin of the human subject where hair growth is desired; and
b. administering a pharmaceutical composition comprising valproic acid or a pharmaceutically acceptable salt, isotopic variant, or solvate thereof

2. The method of claim 1, wherein the area of the skin of the human subject where hair growth is desired is the scalp of the human subject or a part of the scalp of the human subject.

3. The method of claim 1 or 2, wherein the valproic acid or a pharmaceutically acceptable salt thereof is present in an amount of 0.5-30% wt % based on the total weight of the composition.

4. The method of claim 1 or 2, wherein the valproic acid or a pharmaceutically acceptable salt thereof is present in an amount of 2.0-25% wt % based on the total weight of the composition.

5. The method of any one of claims 1-4, wherein the integumental perturbation is performed once a week.

6. The method of any one of claims 1-4, wherein the integumental perturbation is performed twice a month.

7. The method of any one of claim 1-4, wherein the integumental perturbation is performed once a month.

8. The method of any one of claim 1-7, wherein the integumental perturbation is performed for a period of one month, two months, three months, or 4 months.

9. The method of any one of claims 1-7, wherein the integumental perturbation is performed for a period of more than 4 months.

10. The method of any one of claims 1-9, wherein the composition is administered once reepithelialization is completed, or 1, 2, 3, 4, 5, 6, 7, 8, 9, 10, 11, 12, 13, 14, 15, or 16 weeks after integumental perturbation.

11. The method of any one of claims 1-9, wherein the composition is administered once reepithelialization is completed, or at least 2, 4, 8, 10, 12, 14, 16, 18, 20, 22, or 24 hours after integumental perturbation.

12. The method of any one of claims 1-11, wherein the composition is administered before and after integumental perturbation.

13. The method of any one of claims 1-12, wherein the composition is administered for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks.

14. The method of any one of claims 1-13, wherein the composition is administered 1, 2, 3, or 4 times every 1, 2, 3, or 4 days for a period of 1, 2, 3, 4, 5, 6, or 7 days.

15. The method any one of claims 1-14, wherein the composition is administered 1 or 2 times every 1 day for a period of 1, 2, 3, 4, 5, 6, or 7 days.

16. The method of claim 1, wherein the integumental perturbation is performed once a week, the composition is administered at least once a day for at least 5 days following integumental perturbation with the first administration occurring once reepithelialization is completed or at least 24 hours after integumental perturbation, and wherein the integumental perturbation and composition administration is continued for 12 weeks or 16 weeks.

17. The method of claim 16, wherein the composition is administered once a day for 6 days following integumental perturbation.

18. The method of claim 16, wherein the composition is administered twice a day for 6 days following integumental perturbation.

19. The method of claim 1, wherein the integumental perturbation is performed bi-weekly, the composition is administered at least once a day for at least 12 days following integumental perturbation with the first administration occurring once reepithelialization is completed or at least 24 hours after integumental perturbation, and wherein the integumental perturbation and composition administration is continued for 12 weeks or 16 weeks.

20. The method of claim 18, wherein the composition is administered once a day for 13 days following integumental perturbation.

21. The method of claim 18, wherein the composition is administered twice a day for 13 days following integumental perturbation.

22. The method of claim 1, wherein the integumental perturbation is performed once a month, the composition is administered at least once a day for at least 26 days following integumental perturbation with the first administration occurring once reepithelialization is completed or at least 24 hours after integumental perturbation, and wherein the integumental perturbation and composition administration is continued for 12 weeks or 16 weeks.

23. The method of claim 22, wherein the composition is administered once a day for 27 days following integumental perturbation.

24. The method of claim 22, wherein the composition is administered twice a day for 27 days following integumental perturbation.

25. The method of any one of claims 1-24, wherein the integumental perturbation penetrates the skin to a depth of 500 μm to 2.5 mm.

26. The method of any one of claims 1-25, wherein the integumental perturbation is performed by dermabrasion, laser, or controlled integumental perturbation.

27. The method of any one of claims 1-26, wherein the integumental perturbation is performed by dermabrasion.

28. The method of any one of claims 1-27, wherein the integumental perturbation is performed until pinpoint bleeding occurs.

29. The method of any one of claims 1-28, wherein the integumental perturbation is performed by a needling device or drug applicator device.

30. The method of any one of claims 1-29, wherein the integumental perturbation is performed by micro-needling.

31. The method of any one of claims 25-30, wherein the maximum depth of the integumental perturbation is 500, 600, 700, 800, 900, 1000, 1100, 1200, 1300, 1400, 1500, 1600, 1700, 1800, 1900, 2000, 2100, 2200, 2300, 2400 or 2500 μm.

32. The method of any one of claims 25-31, wherein the maximum depth of the integumental perturbation is 700, 800, 900, or 1000 μm.

33. The method of claim 32, wherein the maximum depth of the integumental perturbation is 800 μm.

34. The method of any one of claims 1-33, wherein the composition is administered transdermally.

35. The method of any one of claims 1-33, wherein the composition is administered subcutaneously or externally applied to the skin of the subject.

36. The method of any one of claims 1-33, wherein the composition is administered orally.

37. The method of any one of claims 1-33, wherein the composition is a cream, gel, lotion, emulsion, suspension, oil, non-aqueous solution, aqueous solution, or drop.

38. The method of any one of claims 1-33, wherein the composition is a gel, hydrogel, emulsion, solution, suspension, cream, ointment, dusting powder, dressing, elixir, lotion, suspension, tincture, paste, powder, crystal, foams film, aerosol, irrigation, spray, suppository, stick, bar, ointment, bandage, wound dressing, microdermabrasion or dermabrasion particle, drop, transdermal patch, or dermal patch.

39. The method of any one of claims 1-33, wherein the composition is an aqueous formulation, non-aqueous formulation, ointment, or cream.

40. The method of any one of claims 1-39, wherein the composition is administered for 1, 2, 3 or more months.

41. The method of any one of claims 1-41, wherein the composition is administered by a drug applicator device or cartridge.

42. The method of claim 41, wherein the cartridge contains multiple compounds for simultaneous delivery.

43. The method of any one of claims 1-42, wherein the composition is administered as part of an article of manufacture.

44. The method of claim 43, wherein the article of manufacture is a wound healing dressing.

45. The method of claim 44, wherein the wound healing dressing is a bandage.

46. The method of any one of claims 1-45, wherein the method comprises administering a hair growth promoting agent.

47. The method of any one of claims 1-46, wherein the hair growth promoting agent is not minoxidil, finasteride, dutasteride, fluridil, a spironolactone, a cyproterone acetate, bicalutamide, flutamide, nilutamide, an inhibitor of an androgen receptor, an androgen antagonist, or an antiandrogen.

48. The method of any one of claims 1-47, wherein the method comprises administering an additional active ingredient.

49. The method of claim 48, wherein the method comprises administering an additional active ingredient before, after, or concurrently with administration of the pharmaceutical composition comprising valproic acid or a pharmaceutically acceptable salt, isotopic variant, or solvate thereof.

50. The method of any one of claims 1-49, wherein at 3 months after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more vellus hair compared to immediately before the integumental perturbation.

51. The method of any one of claims 1-50, wherein at 3 months after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more non-vellus hair compared to immediately before the integumental perturbation.

52. The method of any one of claims 1-51, wherein at 3 months after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more terminal hair compared to immediately before the integumental perturbation.

53. The method of any one of claims 1-52, wherein at 2 weeks after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more developing neofollicles compared to immediately before the use of the methods described.

54. The method of any one of claims 1-53, wherein the pharmaceutical composition is administered 1, 2, 3, or more weeks after integumental perturbation.

55. The method of claim 54, wherein the pharmaceutical composition is administered 1 week after integumental perturbation.

56. The method of any one of claims 1-55, wherein the pharmaceutical composition is administered 1 week before integumental perturbation.

57. The method of any one of claims 29-56, wherein the needling device comprises:
a. a sheath assembly comprising a needle array; and
b. a main unit comprising a motor for driving the needle array, wherein the main unit is configured to be fully encapsulated within the sheath assembly so that all parts of the main unit are protected from the outside environment.

58. The method of any one of claims 29-57, wherein the needling device comprises a needling adaptor arranged on a rectangular needle holder in two parallel or substantially parallel rows.

59. The method of any one of claims 29-58, wherein the needling device is a micropen.

60. A course of therapy for promoting hair growth in a human subject, wherein the course comprises performing the method of claim 1 one or more times.

61. The course of claim 60, wherein the course occurs over 1, 2, or 3 months.

62. The course of claim 60 or 61, wherein the course comprises performing integumental perturbation and administering the pharmaceutical composition comprising valproic acid or a pharmaceutically acceptable salt thereof 3, 6, or 12 times.

63. The course of any one of claims 60-62, wherein the course comprises performing integumental perturbation monthly, biweekly, or weekly.

64. The course of any one of claims 60-63, wherein the course comprises administering the pharmaceutical composition comprising valproic acid or a pharmaceutically acceptable salt thereof 1, 2, 3, or 4 times every day for 1, 2, 3, 4, 5, 6, or 7 days.

65. The course of any one of claims 60-64, wherein the course comprises administering the pharmaceutical composition comprising valproic acid or a pharmaceutically acceptable salt thereof 1 or 2 times every day for 1, 2, 3, or 4 weeks.

66. The course of any one of claims 60-65, wherein the course comprises administering the pharmaceutical composition comprising valproic acid or a pharmaceutically acceptable salt thereof 1 or 2 times every day for 1, 2, 3, 4 or more days.
